(19) United States
(12) Patent Application Publication
(10) Pub. No.: US 2021/0249116 A1
(43) Pub. Date: Aug. 12, 2021

(54) Smart Glasses and Wearable Systems for Measuring Food Consumption

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 17/239,960

(22) Filed: Apr. 26, 2021

Related U.S. Application Data
(63) Continuation-in-part of application No. 16/737,052, filed on Jan. 8, 2020.
Application No. 16/737,052 is a continuation-in-part of application No. 16/568,580, filed on Sep. 12, 2019.
Application No. 16/568,580 is a continuation-in-part of application No. 15/963,061, filed on Apr. 25, 2018, now Pat. No. 10,772,559.
Application No. 15/963,061 is a continuation-in-part of application No. 15/431,769, filed on Feb. 14, 2017, now abandoned.
Application No. 16/568,580 is a continuation-in-part of application No. 15/963,061, filed on Apr. 25, 2018, now Pat. No. 10,772,559.
Application No. 15/963,061 is a continuation-in-part of application No. 15/431,769, filed on Feb. 14, 2017, now abandoned.
Application No. 15/963,061 is a continuation-in-part of application No. 14/992,073, filed on Jan. 11, 2016, now abandoned.
Application No. 14/992,073 is a continuation-in-part of application No. 14/550,953, filed on Nov. 22, 2014, now abandoned.
Application No. 15/431,769 is a continuation-in-part of application No. 15/206,215, filed on Jul. 8, 2016, now abandoned.
Application No. 15/206,215 is a continuation-in-part of application No. 14/992,073, filed on Jan. 11, 2016, now abandoned.
Application No. 14/992,073 is a continuation-in-part of application No. 14/330,649, filed on Jul. 14, 2014, now abandoned.
Application No. 15/206,215 is a continuation-in-part of application No. 14/948,308, filed on Nov. 21, 2015, now abandoned.
Application No. 14/992,073 is a continuation-in-part of application No. 14/562,719, filed on Dec. 7, 2014, now Pat. No. 10,130,277.
Application No. 14/562,719 is a continuation-in-part of application No. 13/616,238, filed on Sep. 14, 2012, now abandoned.
Application No. 14/948,308 is a continuation-in-part of application No. 14/550,953, filed on Nov. 22, 2014, now abandoned.
Application No. 14/550,953 is a continuation-in-part of application No. 14/449,387, filed on Aug. 1, 2014, now abandoned.
Application No. 14/449,387 is a continuation-in-part of application No. 14/132,292, filed on Dec. 18, 2013, now Pat. No. 9,442,100.
Application No. 14/132,292 is a continuation-in-part of application No. 13/901,099, filed on May 23, 2013, now Pat. No. 9,254,099.
Application No. 14/330,649 is a continuation-in-part of application No. 13/523,739, filed on Jun. 14, 2012, now Pat. No. 9,042,596.
(60) Provisional application No. 63/171,838, filed on Apr. 7, 2021.
(60) Provisional application No. 62/800,478, filed on Feb. 2, 2019.
(60) Provisional application No. 61/932,517, filed on Jan. 28, 2014.

(51) Int. Cl. G16H 20/60; G06K 9/00; G01N 33/02; G06F 1/16

(52) U.S. Cl. CPC G16H 20/60; G06K 9/00671; G06K 2209/17; G06F 1/163; G01N 33/02

(57) ABSTRACT

This invention is a wearable device or system for measuring food consumption using multiple sensors which are incorporated into smart glasses, a smart watch (or wrist band), or both. These sensors include one or more cameras on the smart glasses, on the smart watch, or both which record food images when eating is detected by a motion sensor, an EMG sensor, and/or a microphone. The smart watch (or wrist band) can also include a spectroscopic sensor to analyze the molecular and/or nutritional composition of food.

### CROSS-REFERENCE TO RELATED APPLICATIONS

This application claims the priority benefit of U.S. provisional patent 63/171,838 filed on 2021 Apr. 7. This application is a continuation in part of U.S. patent application Ser. No. 16/737,052 filed on 2020 Jan. 8. U.S. patent application Ser. No. 16/737,052 was a continuation in part of U.S. patent application Ser. No. 16/568,580 filed on 2019 Sep. 12. U.S. patent application Ser. No. 16/737,052 claimed the priority benefit of U.S. provisional patent application 62/800,478 filed on 2019 Feb. 2. U.S. patent application Ser. No. 16/737,052 was a continuation in part of U.S. patent application Ser. No. 15/963,061 filed on 2018 Apr. 25 which issued as U.S. Pat. No. 10,772,559 on 2020 Sep. 15. U.S. patent application Ser. No. 16/737,052 was a continuation in part of U.S. patent application Ser. No. 15/431,769 filed on 2017 Feb. 14. U.S. patent application Ser. No. 16/568,580 was a continuation in part of U.S. patent application Ser. No. 15/963,061 filed on 2018 Apr. 25 which issued as U.S. Pat. No. 10,772,559 on 2020 Sep. 15. U.S. patent application Ser. No. 16/568,580 was a continuation in part of U.S. patent application Ser. No. 15/431,769 filed on 2017 Feb. 14.

U.S. patent application Ser. No. 15/963,061 was a continuation in part of U.S. patent application Ser. No. 14/992,073 filed on 2016 Jan. 11. U.S. patent application Ser. No. 15/963,061 was a continuation in part of U.S. patent application Ser. No. 14/550,953 filed on 2014 Nov. 22. U.S. patent application Ser. No. 15/431,769 was a continuation in part of U.S. patent application Ser. No. 15/206,215 filed on 2016 Jul. 8. U.S. patent application Ser. No. 15/431,769 was a continuation in part of U.S. patent application Ser. No. 14/992,073 filed on 2016 Jan. 11. U.S. patent application Ser. No. 15/431,769 was a continuation in part of U.S. patent application Ser. No. 14/330,649 filed on 2014 Jul. 14. U.S. patent application Ser. No. 15/206,215 was a continuation in part of U.S. patent application Ser. No. 14/948,308 filed on 2015 Nov. 21. U.S. patent application Ser. No. 14/992,073 was a continuation in part of U.S. patent application Ser. No. 14/562,719 filed on 2014 Dec. 7 which issued as U.S. Pat. No. 10,130,277 on 2018 Nov. 20. U.S. patent application Ser. No. 14/992,073 was a continuation in part of U.S. patent application Ser. No. 13/616,238 filed on 2012 Sep. 14.

U.S. patent application Ser. No. 14/948,308 was a continuation in part of U.S. patent application Ser. No. 14/550,953 filed on 2014 Nov. 22. U.S. patent application Ser. No. 14/948,308 was a continuation in part of U.S. patent application Ser. No. 14/449,387 filed on 2014 Aug. 1. U.S. patent application Ser. No. 14/948,308 was a continuation in part of U.S. patent application Ser. No. 14/132,292 filed on 2013 Dec. 18 which issued as U.S. Pat. No. 9,442,100 on 2016 Sep. 13. U.S. patent application Ser. No. 14/948,308 was a continuation in part of U.S. patent application Ser. No. 13/901,099 filed on 2013 May 23 which issued as U.S. Pat. No. 9,254,099 on 2016 Feb. 9. U.S. patent application Ser. No. 14/562,719 claimed the priority benefit of U.S. provisional patent application 61/932,517 filed on 2014 Jan. 28. U.S. patent application Ser. No. 14/330,649 was a continuation in part of U.S. patent application Ser. No. 13/523,739 filed on 2012 Jun. 14 which issued as U.S. Pat. No. 9,042,596 on 2015 May 26.

The entire contents of these applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to wearable devices for measuring food consumption.

### INTRODUCTION

Many health problems are caused by poor nutrition. Many people consume too much unhealthy food or not enough healthy food. Although there are complex behavioral reasons for poor dietary habits, better nutritional monitoring and awareness concerning the types and quantities of food consumed can help people to improve their dietary habits and health. Information concerning the types and quantities of food consumed can be part of a system that provides constructive feedback and/or incentives to help people improve their nutritional intake. People can try to track the types and quantities of food consumed without technical assistance. Their unassisted estimates of the types and quantities of consumed food can be translated into types and quantities of nutrients consumed. However, such unassisted tracking can be subjective. Also, such unassisted tracking can be particularly challenging for non-standardized food items such as food prepared in an ad hoc manner at restaurants or in homes. It would be useful to have a relatively-unobtrusive device which can help people to accurately track the types and quantities of food which they consume.

### Review of the Relevant Art

The following art is relevant and prior to the application date of this application, but not all of it is prior to the application dates of parent applications for which priority and/or continuation are claimed by this application. U.S. patent application publications 20090012433 (Fernstrom et al., Jan. 8, 2009, “Method, Apparatus and System for Food Intake and Physical Activity Assessment”), 20130267794 (Fernstrom et al., Oct. 10, 2013, “Method, Apparatus and System for Food Intake and Physical Activity Assessment”), and 20180348187 (Fernstrom et al., Dec. 6, 2018, “Method, Apparatus and System for Food Intake and Physical Activity Assessment”), as well as U.S. Pat. No. 9,198,621 (Fernstrom et al., Dec. 1, 2015, “Method, Apparatus and System for Food Intake and Physical Activity Assessment”) and U.S. Pat. No. 10,006,896 (Fernstrom et al., Jun. 26, 2018, “Method, Apparatus and System for Food Intake and Physical Activity Assessment”), disclose wearable buttons and necklaces for monitoring eating with cameras. U.S. Pat. No. 10,900,943 (Fernstrom et al, Jan. 26, 2021, “Method, Apparatus and System for Food Intake and Physical Activity Assessment”) discloses monitoring food consumption using a wearable device with two video cameras and an infrared sensor.

U.S. patent application publication 20160073953 (Sazonov et al., Mar. 17, 2016, “Food Intake Monitor”) discloses monitoring food consumption using a wearable device with a jaw motion sensor and a hand gesture sensor. U.S. patent application publication 20180242908 (Sazonov et al., Aug. 30, 2018, “Food Intake Monitor”) and U.S. Pat. No. 10,736,566 (Sazonov, Aug. 11, 2020, “Food Intake Monitor”) disclose monitoring food consumption using an ear-worn device or eyeglasses with a pressure sensor and accelerometer.

U.S. patent application publications 20160299061 (Goldring et al., 10/13/2016, “Spectrometry Systems, Methods, and Applications”), 20170160131 (Goldring et al., Jun. 8, 2017, “Spectrometry Systems, Methods, and Applications”), 20180085003 (Goldring et al., Mar. 29, 2018, “Spectrometry Systems, Methods, and Applications”), 20180120155 (Rosen et al., May 3, 2018, “Spectrometry Systems, Methods, and Applications”), and 20180180478 (Goldring et al., Jun. 28, 2018, “Spectrometry Systems, Methods, and Applications”) disclose a handheld spectrometer to measure the spectra of objects. U.S. patent application publication 20180136042 (Goldring et al., May 17, 2018, “Spectrometry System with Visible Aiming Beam”) discloses a handheld spectrometer with a visible aiming beam. U.S. patent application publication 20180252580 (Goldring et al., Sep. 6, 2018, “Low-Cost Spectrometry System for End-User Food Analysis”) discloses a compact spectrometer that can be used in mobile devices such as smart phones. U.S. patent application publication 20190033130 (Goldring et al., Jan. 31, 2019, “Spectrometry Systems, Methods, and Applications”) discloses a hand held spectrometer with wavelength multiplexing. U.S. patent application publication 20190033132 (Goldring et al., Jan. 31, 2019, “Spectrometry System with Decreased Light Path”) discloses a spectrometer with a plurality of isolated optical channels.

U.S. patent application publications 20190244541 (Hadad et al., Aug. 8, 2019, “Systems and Methods for Generating Personalized Nutritional Recommendations”), 20140255882 (Hadad et al., Sep. 11, 2014, “Interactive Engine to Provide Personal Recommendations for Nutrition, to Help the General Public to Live a Balanced Healthier Lifestyle”), and 20190290172 (Hadad et al., Sep. 26, 2019, “Systems and Methods for Food Analysis, Personalized Recommendations, and Health Management”) disclose methods to provide nutrition recommendations based on a person's preferences, habits, medical and activity.

U.S. patent application publication 20190333634 (Vleugels et al., Oct. 31, 2019, “Method and Apparatus for Tracking of Food Intake and Other Behaviors and Providing Relevant Feedback”), 20170220772 (Vleugels et al., Aug. 3, 2017, “Method and Apparatus for Tracking of Food Intake and Other Behaviors and Providing Relevant Feedback”), and 20180300458 (Vleugels et al., Oct. 18, 2018, “Method and Apparatus for Tracking of Food Intake and Other Behaviors and Providing Relevant Feedback”), as well as U.S. Pat. No. 10,102,342 (Vleugels et al., Oct. 16, 2018, “Method and Apparatus for Tracking of Food Intake and Other Behaviors and Providing Relevant Feedback”) and U.S. Pat. No. 10,373,716 (Vleugels et al., Aug. 6, 2019, “Method and Apparatus for Tracking of Food Intake and Other Behaviors and Providing Relevant Feedback”), disclose a method for detecting, identifying, analyzing, quantifying, tracking, processing and/or influencing food consumption.

U.S. patent application publication 20200294645 (Vleugels, Sep. 17, 2020, “Gesture-Based Detection of a Physical Behavior Event Based on Gesture Sensor Data and Supplemental Information from at Least One External Source”) discloses an automated medication dispensing system which recognizes gestures. U.S. patent application publication 20200381101 (Vleugels, Dec. 3, 2020, “Method and Apparatus for Tracking of Food Intake and Other Behaviors and Providing Relevant Feedback”) discloses methods for detecting, identifying, analyzing, quantifying, tracking, processing and/or influencing, related to the intake of food, eating habits, eating patterns, and/or triggers for food intake events, eating habits, or eating patterns. U.S. Pat. No. 10,790,054 (Vleugels et al., Sep. 29, 2020, “Method and Apparatus for Tracking of Food Intake and Other Behaviors and Providing Relevant Feedback”) discloses a computer-based method of detecting gestures.

U.S. Pat. No. 10,901,509 (Aimone et al., Jan. 26, 2021, “Wearable Computing Apparatus and Method”) discloses a wearable computing device comprising at least one brainwave sensor. U.S. patent application publication 20160163037 (Dehais et al., Jun. 9, 2016, “Estimation of Food Volume and Carbs”) discloses an image-based food identification system including a projected light pattern. U.S. patent application publication 20170249445 (Devries et al., Aug. 31, 2017, “Portable Devices and Methods for Measuring Nutritional Intake”) discloses a nutritional intake monitoring system with biosensors.

U.S. patent application publication 20160140869 (Kuwahara et al., May 19, 2016, “Food Intake Controlling Devices and Methods”) discloses image-based technologies for controlling food intake. U.S. patent application publication 20150302160 (Muthukumar et al., Oct. 22, 2015, “Method and Apparatus for Monitoring Diet and Activity”) discloses a method and device for analyzing food with a camera and a spectroscopic sensor. U.S. Pat. No. 10,249,214 (Novotny et al., Apr. 2, 2019, “Personal Wellness Monitoring System”) discloses monitoring health and wellness using a camera. U.S. patent application publication 20180005545 (Pathak et al., Jan. 4, 2018, “Assessment of Nutrition Intake Using a Handheld Tool”) discloses a smart food utensil for measuring food mass.

U.S. patent application publication 20160091419 (Watson et al., Mar. 31, 2016, “Analyzing and Correlating Spectra, Identifying Samples and Their Ingredients, and Displaying Related Personalized Information”) discloses a spectral analysis method for food analysis. U.S. patent application publications 20170292908 (Wilk et al., Oct. 12, 2017, “Spectrometry System Applications”) and 20180143073 (Goldring et al., May 24, 2018, “Spectrometry System Applications”) disclose a spectrometer system to determine spectra of an object. U.S. patent application publication 20170193854 (Yuan et al., 2016 Jan. 5, “Smart Wearable Device and Health Monitoring Method”) discloses a wearable device with a camera to monitor eating. U.S. Pat. No. 10,058,283 (Zerick et al., 2016 Apr. 6, “Determining Food Identities with Intra-Oral Spectrometer Devices”) discloses an intra-oral device for food analysis.

The following are relevant published articles. Full bibliographic information for these articles is included in the Information Disclosure Statement (IDS) accompanying this application. (Amft et al, 2005, “Detection of Eating and Drinking Arm Gestures Using Inertial Body-Worn Sensors”) discloses eating detection by analyzing arm gestures. (Bedri et al, 2015, “Detecting Mastication: A Wearable Approach”; access to abstract only) discloses eating detection using an ear-worn devices with a gyroscope and proximity sensors. (Bedri et al, 2017, “EarBit: Using Wearable Sensors to Detect Eating Episodes in Unconstrained Environments”) discloses eating detection using an ear-worn device with inertial, optical, and acoustic sensors. (Bedri et al, 2020a, “FitByte: Automatic Diet Monitoring in Unconstrained Situations Using Multimodal Sensing on Eyeglasses”) discloses food consumption monitoring using a device with a motion sensor, an infrared sensor, and a camera which is attached to eyeglasses. (Bell et al, 2020, “Automatic, Wearable-Based, In-Field Eating Detection Approaches for Public Health Research: A Scoping Review”) reviews wearable sensors for eating detection.

(Bi et al, 2016, “AutoDietary: A Wearable Acoustic Sensor System for Food Intake Recognition in Daily Life”) discloses eating detection using a neck-worn device with sound sensors. (Bi et al, 2017, “Toward a Wearable Sensor for Eating Detection”) discloses eating detection using ear-worn and neck-worn devices with sound sensors and EMG sensors. (Bi et al, 2018, “Auracle: Detecting Eating Episodes with an Ear-Mounted Sensor”) discloses eating detection using an ear-worn device with a microphone. (Borrell, 2011, “Every Bite You Take”) discloses food consumption monitoring using a neck-worn device with GPS, a microphone, an accelerometer, and a camera. (Brenna et al, 2019, “A Survey of Automatic Methods for Nutritional Assessment) reviews automatic methods for nutritional assessment. (Chun et al, 2018, “Detecting Eating Episodes by Tracking Jawbone Movements with a Non-Contact Wearable Sensor”) discloses eating detection using a necklace with an accelerometer and range sensor.

(Chung et al, 2017, “A Glasses-Type Wearable Device for Monitoring the Patterns of Food Intake and Facial Activity”) discloses eating detection using a force-based chewing sensor on eyeglasses. (Dimitratos et al, 2020, “Wearable Technology to Quantify the Nutritional Intake of Adults: Validation Study”) discloses high variability in food consumption monitoring using only a wristband with a motion sensor. (Dong et al, 2009, “A Device for Detecting and Counting Bites of Food Taken by a Person During Eating”) discloses bite counting using a wrist-worn orientation sensor. (Dong et al, 2011, “Detecting Eating Using a Wrist Mounted Device During Normal Daily Activities”) discloses eating detection using a watch with a motion sensor. (Dong et al, 2012b, “A New Method for Measuring Meal Intake in Humans via Automated Wrist Motion Tracking”) discloses bite counting using a wrist-worn gyroscope. (Dong et al, 2014, “Detecting Periods of Eating During Free-Living by Tracking Wrist Motion”) discloses eating detection using a wrist-worn device with motion sensors.

(Farooq et al, 2016, “A Novel Wearable Device for Food Intake and Physical Activity Recognition”) discloses eating detection using eyeglasses with a piezoelectric strain sensor and an accelerometer. (Farooq et al, 2017, “Segmentation and Characterization of Chewing Bouts by Monitoring Temporalis Muscle Using Smart Glasses With Piezoelectric Sensor”) discloses chew counting using eyeglasses with a piezoelectric strain sensor. (Fontana et al, 2014, “Automatic Ingestion Monitor: A Novel Wearable Device for Monitoring of Ingestive Behavior”) discloses food consumption monitoring using a device with a jaw motion sensor, a hand gesture sensor, and an accelerometer. (Fontana et al, 2015, “Energy Intake Estimation from Counts of Chews and Swallows”) discloses counting chews and swallows using wearable sensors and video analysis. (Jasper et al, 2016, “Effects of Bite Count Feedback from a Wearable Device and Goal-Setting on Consumption in Young Adults”) discloses the effect of feedback based on bite counting.

(Liu et al, 2012, “An Intelligent Food-Intake Monitoring System Using Wearable Sensors”) discloses food consumption monitoring using an ear-worn device with a microphone and camera. (Magrini et al, 2017, “Wearable Devices for Caloric Intake Assessment: State of Art and Future Developments”) reviews wearable devices for automatic recording of food consumption. (Makeyev et al, 2012, “Automatic Food Intake Detection Based on Swallowing Sounds”) discloses swallowing detection using wearable sound sensors. (Merck et al, 2016, “Multimodality Sensing for Eating Recognition”; access to abstract only) discloses eating detection using eyeglasses and smart watches on each wrist, combining motion and sound sensors.

(Mirtchouk et al, 2016, “Automated Estimation of Food Type and Amount Consumed from Body-Worn Audio and Motion Sensors”; access to abstract only) discloses food consumption monitoring using in-ear audio plus head and wrist motion. (Mirtchouk et al, 2017, “Recognizing Eating from Body-Worn Sensors: Combining Free-Living and Laboratory Data”) discloses eating detection using head-worn and wrist-worn motion sensors and sound sensors. (O'Loughlin et al, 2013, “Using a Wearable Camera to Increase the Accuracy of Dietary Analysis”) discloses food consumption monitoring using a combination of a wearable camera and self-reported logging. (Prioleau et al, 2017, “Unobtrusive and Wearable Systems for Automatic Dietary Monitoring”) reviews wearable and hand-held approaches to dietary monitoring. (Rahman et al, 2015, “Unintrusive Eating Recognition Using Google Glass”) discloses eating detection using eyeglasses with an inertial motion sensor.

(Sazonov et al, 2008, “Non-Invasive Monitoring of Chewing and Swallowing for Objective Quantification of Ingestive Behavior”) discloses counting chews and swallows using ear-worn and/or neck-worn strain and sound sensors. (Sazonov et al, 2009, “Toward Objective Monitoring of Ingestive Behavior in Free-Living Population”) discloses counting chews and swallows using strain sensors. (Sazonov et al, 2010a, “The Energetics of Obesity: A Review: Monitoring Energy Intake and Energy Expenditure in Humans”) reviews devices for monitoring food consumption. (Sazonov et al, 2010b, “Automatic Detection of Swallowing Events by Acoustical Means for Applications of Monitoring of Ingestive Behavior”) discloses swallowing detection using wearable sound sensors. (Sazonov et al, 2012, “A Sensor System for Automatic Detection of Food Intake Through Non-Invasive Monitoring of Chewing”) discloses eating detection using a wearable piezoelectric strain gauge.

(Schiboni et al, 2018, “Automatic Dietary Monitoring Using Wearable Accessories”) reviews wearable devices for dietary monitoring. (Sen et al, 2018, “Annapurna: Building a Real-World Smartwatch-Based Automated Food Journal”; access to abstract only) discloses food consumption monitoring using a smart watch with a motion sensor and a camera. (Sun et al, 2010, “A Wearable Electronic System for Objective Dietary Assessment”) discloses food consumption monitoring using a wearable circular device with earphones, microphones, accelerometers, or skin-surface electrodes. (Tamura et al, 2016, “Review of Monitoring Devices for Food Intake”) reviews wearable devices for eating detection and food consumption monitoring. (Thomaz et al, 2013, “Feasibility of Identifying Eating Moments from First-Person Images Leveraging Human Computation”) discloses eating detection through analysis of first-person images. (Thomaz et al, 2015, “A Practical Approach for Recognizing Eating Moments with Wrist-Mounted Inertial Sensing”) discloses eating detection using a smart watch with an accelerometer.

(Vu et al, 2017, “Wearable Food Intake Monitoring Technologies: A Comprehensive Review”) reviews sensing platforms and data analytic approaches to solve the challenges of food-intake monitoring, including ear-based chewing and swallowing detection systems and wearable cameras. (Young, 2020, “FitByte Uses Sensors on Eyeglasses to Automatically Monitor Diet: CMU Researchers Propose a Multimodal System to Track Foods, Liquid Intake”) discloses food consumption monitoring using a device with a motion sensor, an infrared sensor, and a camera which is attached to eyeglasses. (Zhang et al, 2016, “Diet Eyeglasses: Recognising Food Chewing Using EMG and Smart Eyeglasses”; access to abstract only) discloses eating detection using eyeglasses with EMG sensors. (Zhang et al, 2018a, “Free-Living Eating Event Spotting Using EMG-Monitoring Eyeglasses”; access to abstract only) discloses eating detection using eyeglasses with EMG sensors. (Zhang et al, 2018b, “Monitoring Chewing and Eating in Free-Living Using Smart Eyeglasses”) discloses eating detection using eyeglasses with EMG sensors.

### SUMMARY OF THE INVENTION

As evidenced by the preceding review of relevant art, there has been an increase in research on wearable devices for measuring food consumption during the past several years. Many of the devices in the relevant art detect when a person is eating food or drinking, but are not very good at measuring how much food the person eats or how much beverage the person drinks, often crudely estimating food or beverage quantity by the number of hand motions, bites, and/or swallows. Other devices which include a camera and analyze food images are better at measuring food or beverage quantities, but a camera which constantly records images can be intrusive on privacy. Also, camera images do not provide good information about the nutritional content of non-standardized (e.g. home-prepared) meals. The wearable innovative devices and systems for measuring food consumption which are disclosed herein address these limitations of the prior art.

This invention is a wearable device or system for measuring food consumption using multiple sensors which are incorporated into smart glasses, a smart watch (or wrist band), or both. These sensors include one or more cameras on the smart glasses, on the smart watch, or both which are activated to record food images when eating is detected by a motion sensor, EMG sensor, and/or microphone. In some variations of this invention, the smart watch (or wrist band) also includes a spectroscopic sensor to analyze the molecular and/or nutritional composition of food.

### BRIEF DESCRIPTION OF THE FIGURES

FIG. 1 shows smart eyewear for measuring food consumption with a camera.

FIG. 2 shows smart eyewear for measuring food consumption with a camera activated by chewing.

FIG. 3 shows smart eyewear for measuring food consumption with a camera activated by chewing and hand-to-mouth proximity.

FIG. 4 shows a smart watch or wrist band for measuring food consumption with an eating-related motion sensor.

FIG. 5 shows a smart watch or wrist band for measuring food consumption with a camera activated by eating-related motion.

FIG. 6 shows a smart watch or wrist band for measuring food consumption with an eating-related motion sensor and a spectroscopic sensor.

FIG. 7 shows a smart watch or wrist band for measuring food consumption with a camera activated by eating-related motion, and also a spectroscopic sensor.

FIG. 8 shows a wearable system for measuring food consumption with an eyewear camera activated by eating-related wrist motion.

FIG. 9 shows a wearable system for measuring food consumption with an eyewear camera and a wrist-based camera activated by eating-related wrist motion.

FIG. 10 shows a wearable system for measuring food consumption with an eyewear camera activated by eating-related wrist motion, and also a spectroscopic sensor.

FIG. 11 shows a wearable system for measuring food consumption with an eyewear camera and a wrist-based camera activated by eating-related wrist motion, and also a spectroscopic sensor.

FIG. 12 shows a wearable system for measuring food consumption with an eyewear camera activated by eating-related wrist motion and chewing.

FIG. 13 shows a wearable system for measuring food consumption with an eyewear camera and a wrist-based camera activated by eating-related wrist motion and chewing.

FIG. 14 shows a wearable system for measuring food consumption with an eyewear camera activated by eating-related wrist motion and chewing, and also a spectroscopic sensor.

FIG. 15 shows a wearable system for measuring food consumption with an eyewear camera and a wrist-based camera activated by eating-related wrist motion and chewing, and also a spectroscopic sensor.

FIG. 16 shows a wearable system for measuring food consumption with an eyewear camera activated by eating-related wrist motion, chewing, and hand-to-mouth proximity.

FIG. 17 shows a wearable system for measuring food consumption with an eyewear camera and a wrist-worn camera activated by eating-related wrist motion, chewing, and hand-to-mouth proximity.

FIG. 18 shows a wearable system for measuring food consumption with an eyewear camera activated by eating-related wrist motion, chewing, and hand-to-mouth proximity, and also a spectroscopic sensor.

FIG. 19 shows a wearable system for measuring food consumption with an eyewear camera and a wrist-worn camera activated by eating-related wrist motion, chewing, and hand-to-mouth proximity, and also a spectroscopic sensor.

### DETAILED DESCRIPTION OF THE FIGURES

In an example, a wearable food consumption monitoring device can comprise eyeglasses with one or more automatic food imaging members (e.g. cameras), wherein images recorded by the cameras are automatically analyzed to estimate the types and quantities of food consumed by a person. In an example, one or more cameras can start recording images when they are triggered by food consumption detected by analysis of data from one or more sensors selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, infrared sensor, spectroscopy sensor, electrogoniometer, chewing sensor, swallowing sensor, temperature sensor, and pressure sensor.

In an example, a device can comprise eyeglasses which further comprise one or more automatic food imaging members (e.g. cameras). Pictures taken by an imaging member can be automatically analyzed in order to estimate the types and quantities of food which are consumed by a person. Food can refer to beverages as well as solid food. An automatic imaging member can take pictures when it is activated (triggered) by food consumption based on data collected by one or more sensors selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallowing sensor, temperature sensor, and pressure sensor. In an example, when data from one or more sensors indicates that a person is probably consuming food, then this can activate (trigger) an imaging member to start taking pictures and/or recording images.

In an example, eyeglasses to monitor food consumption can include a camera which records images along an imaging vector which points toward a person's mouth. In an example, a camera can record images of a person's mouth and the interaction between food and the person's mouth. Interaction between food and a person's mouth can include biting, chewing, and/or swallowing. In an example, eyeglasses for monitoring food consumption can include a camera which records images along an imaging vector which points toward a reachable food source. In an example, eyeglasses can include two cameras: a first camera which records images along an imaging vector which points toward a person's mouth and a second camera which records images along an imaging vector which points toward a reachable food source.

In an example, a device can comprise at least two cameras or other imaging members. A first camera can take pictures along an imaging vector which points toward a person's mouth while the person eats. A second camera can take pictures along an imaging vector which points toward a reachable food source. In an example, this device can comprise one or more imaging members that take pictures of: food at a food source; a person's mouth; and interaction between food and the person's mouth. Interaction between the person's mouth and food can include biting, chewing, and swallowing. In an example, utensils or beverage-holding members may be used as intermediaries between the person's hand and food. In an example, this invention can comprise an imaging device that automatically takes pictures of the interaction between food and the person's mouth as the person eats. In an example, this device can comprise a wearable device that takes pictures of a reachable food source that is located in front of a person. In an example, such a device can track the location of, and take pictures of, a person's mouth track the location of, and take pictures of, a person's hands; and scan for, and take pictures of, reachable food sources nearby.

In an example, a system for food consumption monitoring can include eyeglasses and a wrist-worn device (e.g. smart watch) which are in electromagnetic communication with each other. In an example, a system for food consumption monitoring can comprise eyeglasses and a wrist-worn motion sensor. In an example, a wrist-worn motion sensor can detect a pattern of hand and/or arm motion which is associated with food consumption. In an example, this pattern of hand and/or arm motion can comprise: hand movement toward a reachable food source; hand movement up to a person's mouth; lateral motion and/or hand rotation to bring food into the mouth; and hand movement back down to the original level. In an example, a food consumption monitoring device can continually track the location of a person's hand to detect when it comes near the person's mouth and/or grasps a reachable food source.

In an example, an imaging member can automatically start taking pictures and/or recording images when data from a wrist-worn motion sensor shows a pattern of hand and/or arm motion which is generally associated with food consumption. In an example, this pattern of hand and/or arm motion can comprise: hand movement toward a reachable food source; hand movement up to a person's mouth; lateral motion and/or hand rotation to bring food into the mouth; and hand movement back down to the original level. In an example, electronically-functional eyewear can be in wireless communication with a motion sensor which is worn on a person's wrist, finger, hand, or arm. In an example, this motion sensor can detect hand, finger, wrist, and/or arm movements which indicate that a person is preparing food for consumption and/or bringing food up to their mouth.

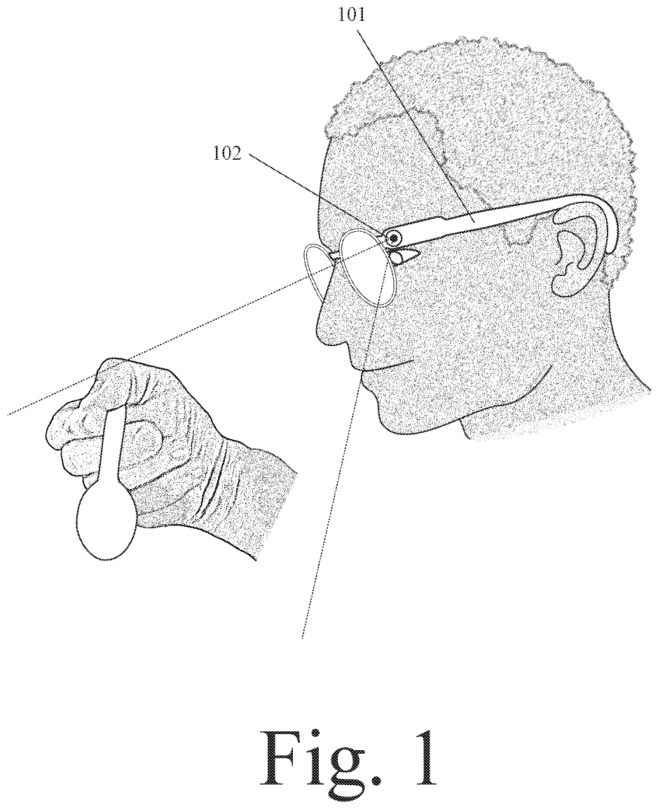

FIG. 1 shows an example of smart eyewear for measuring food consumption comprising: an eyewear frame 101 worn by a person; and a camera 102 on the eyewear frame which records food images when activated. In an example, eyewear can be a pair of eyeglasses. In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear.

In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

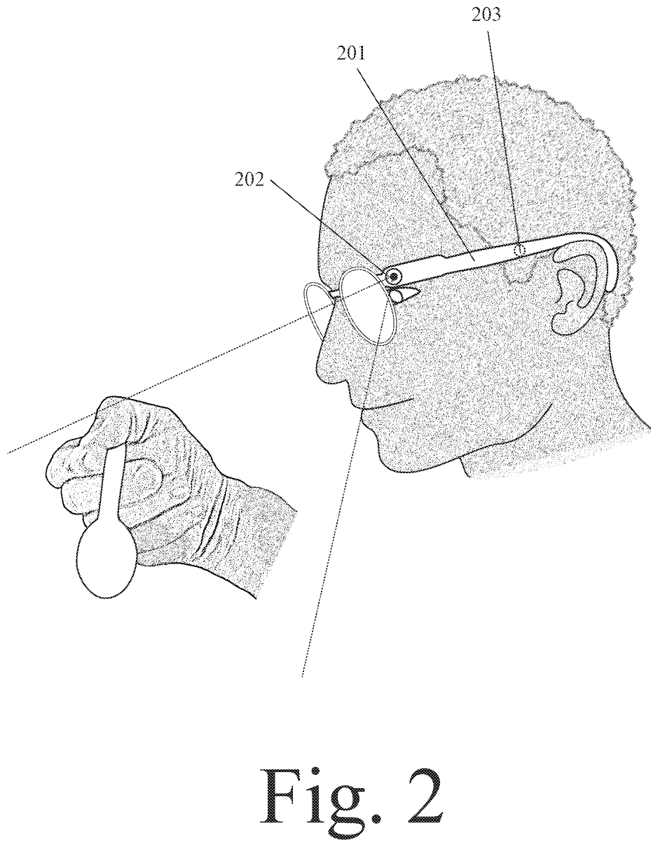

FIG. 2 shows an example of smart eyewear for measuring food consumption comprising: an eyewear frame 201 worn by a person; a camera 202 on the eyewear frame which records food images when activated; and a chewing sensor 203 on the eyewear frame which detects when the person eats, wherein the camera is activated to record food images when data from the chewing sensor indicates that the person is eating. In an example, eyewear can be a pair of eyeglasses. In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear.

In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, a chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

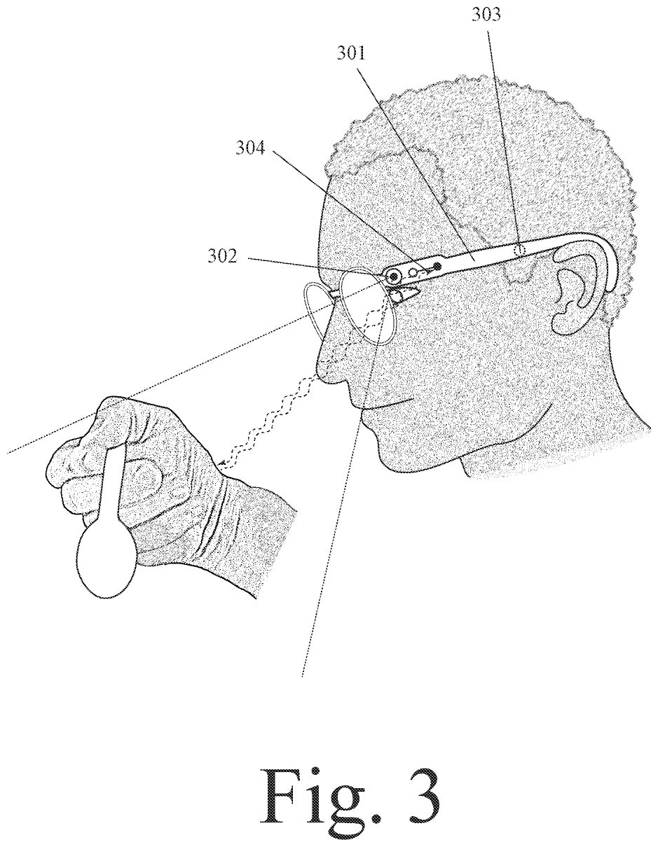

FIG. 3 shows an example of smart eyewear for measuring food consumption comprising: an eyewear frame 301 worn by a person; a camera 302 on the eyewear frame which records food images when activated; a chewing sensor 303 on the eyewear frame which detects when the person eats; and a proximity sensor 304 on the eyewear frame which uses infrared light to detect when a person eats by detecting when an object (such as the person's hand) is near the person's mouth, wherein the camera is activated to record food images when data from the chewing sensor and/or data from the proximity sensor indicate that the person is eating. In an example, eyewear can be a pair of eyeglasses.

In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, a chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

In an example, a proximity sensor can direct a beam of infrared light toward space in front of the person's mouth. This beam is reflected back toward the proximity sensor when an object (such as the person's hand or a food utensil) is in front of the person's mouth. In an example, the camera can be activated by the proximity sensor to confirm that the person's hand is bringing food up to their mouth, not to brush their teeth, cough, or some other hand-near-mouth activity. In an example, joint analysis of data from the chewing sensor and data from the proximity sensor can provide more accurate detection of eating than data from either sensor alone or separate analysis of data from both sensors.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

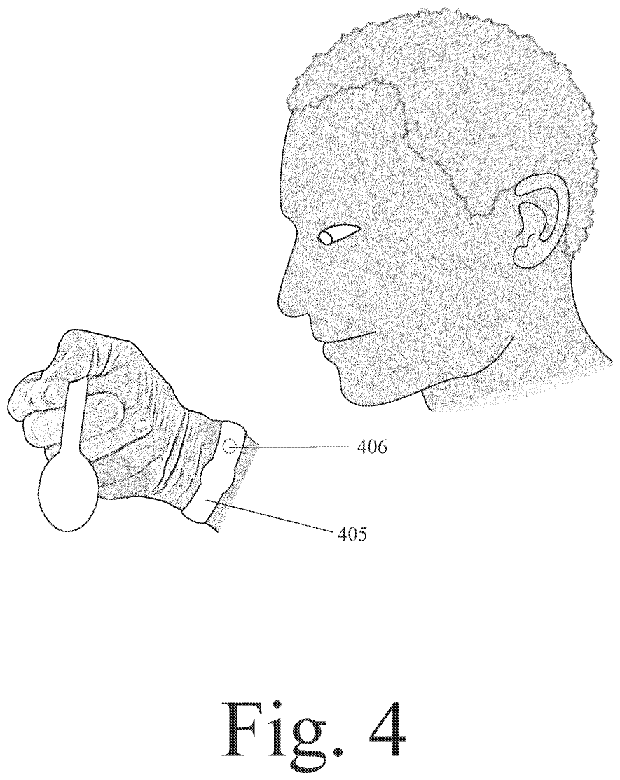

FIG. 4 shows an example of a smart watch, wrist band, or watch band for measuring food consumption comprising: a smart watch (or wrist band) 405 worn by a person; and a motion sensor 406 (e.g. accelerometer and/or gyroscope) on the smart watch (or wrist band), wherein the motion sensor is used to measure the person's food consumption.

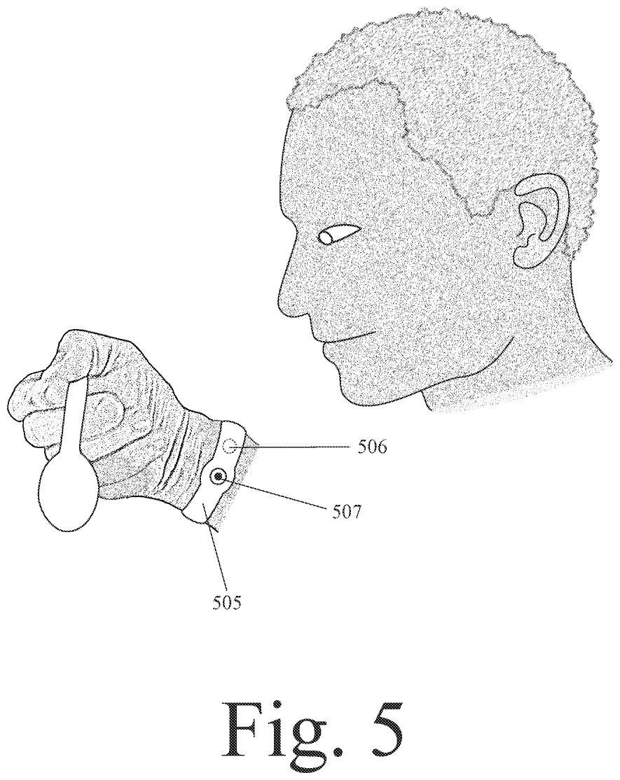

FIG. 5 shows an example of a smart watch, wrist band, or watch band for measuring food consumption comprising: a smart watch (or wrist band) 505 worn by a person; a motion sensor 506 (e.g. accelerometer and/or gyroscope) on the smart watch (or wrist band); and a camera 507 on the smart watch (or wrist band), wherein the camera is activated to record food images when data from the motion sensor indicates that the person is eating. In an example, a camera can be located on the anterior side of a person's wrist (opposite the traditional location of a watch face housing). Alternatively, a camera can be on a watch face housing. In an example, there can be two cameras on a smart watch, wrist band, or watch band to record images of nearby food, hand-to-food interactions, and hand-to-mouth interactions. In an example, one camera can be on the anterior side of a person's wrist and one camera can be on the posterior side of the person's wrist (e.g. on a watch face housing). In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

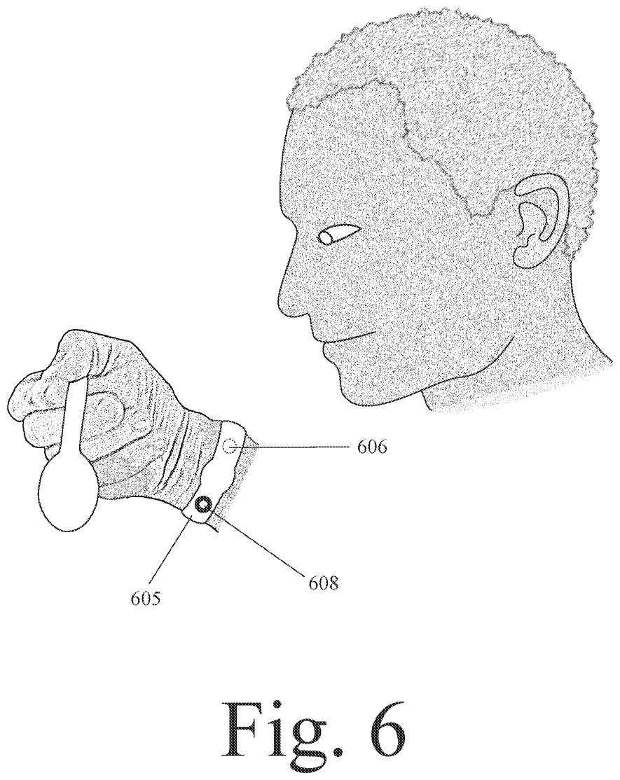

FIG. 6 shows an example of a smart watch, wrist band, or watch band for measuring food consumption comprising: a smart watch (or wrist band) 605 worn by a person; a motion sensor 606 (e.g. accelerometer and/or gyroscope) on the smart watch (or wrist band); and a spectroscopic sensor 608 on the smart watch (or wrist band) which analyzes the molecular and/or nutritional composition of food, wherein the spectroscopic sensor is activated when data from the motion sensor indicates that the person is eating. In another example, instead of the spectroscopic sensor being triggered automatically, the person can be prompted to take a spectroscopic scan of food when the motion sensor indicates that the person is eating. In an example, a person can take a spectroscopic scan of food by waving their hand over food (like Obi-Wan Kenobi). In an example, a spectroscopic sensor can be located on the anterior side of the person's wrist (opposite the traditional location of a watch face). Alternatively, a spectroscopic sensor can be located on the watch face housing. In an example, a spectroscopic sensor can emit light away from the outer surface of a smart watch (or wrist band) and toward food. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

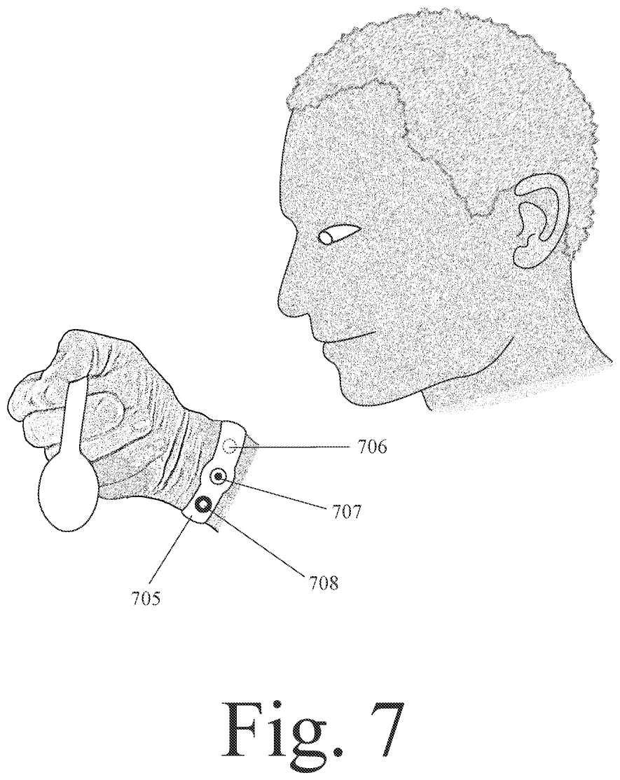

FIG. 7 shows an example of a smart watch, wrist band, or watch band for measuring food consumption comprising: a smart watch (or wrist band) 705 worn by a person; a motion sensor 706 (e.g. accelerometer and/or gyroscope) on the smart watch (or wrist band); a camera 707 on the smart watch (or wrist band), wherein the camera is activated to record food images when data from the motion sensor indicates that the person is eating; and a spectroscopic sensor 708 on the smart watch (or wrist band) which analyzes the molecular and/or nutritional composition of food, wherein the spectroscopic sensor is activated to record food images when data from the motion sensor indicates that the person is eating. In another example, instead of the spectroscopic sensor being triggered automatically, the person can be prompted to take a spectroscopic scan of food when the motion sensor indicates that the person is eating. In an example, a person can take a spectroscopic scan of food by waving their hand over food. In an example, a spectroscopic sensor can emit light away from the outer surface of a smart watch (or wrist band) and toward food. In an example, the spectroscopic sensor can emit and receive near-infrared light.

In an example, a camera on a smart watch (or wrist band) can be located on the anterior side of the person's wrist (opposite the traditional location of a watch face). Alternatively, a camera can be on a watch face housing. In an example, there can be two cameras on a smart watch, wrist band, or watch band to record images of nearby food, hand-to-food interactions, and hand-to-mouth interactions. In an example, one camera can be on the anterior side of a person's wrist and one camera can be on the posterior side of the person's wrist (e.g. on a watch face housing). In an example, one camera can be on a first lateral side of a person's wrist and another camera can be on the opposite lateral side of the person's wrist, so that one camera tends to record images of nearby food and the other camera tends to record images of the person's mouth as the person eats. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

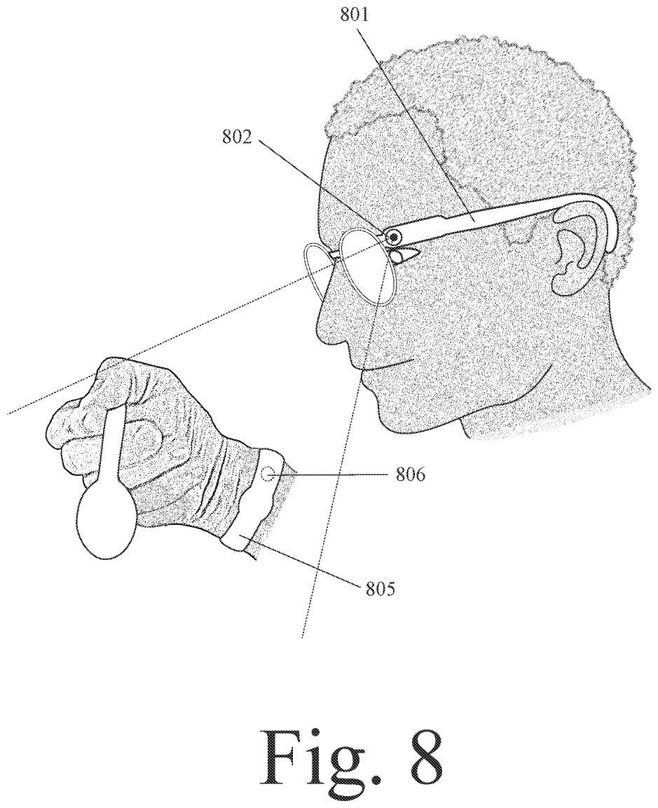

FIG. 8 shows an example of a wearable system for measuring food consumption comprising: an eyewear frame 801 worn by a person; a camera 802 on the eyewear frame which records food images when activated; a smart watch (or wrist band) 805 worn by the person; and a motion sensor 806 (e.g. accelerometer and/or gyroscope) on the smart watch (or wrist band), wherein the camera is activated to record food images when data from the motion sensor indicates that the person is eating. In an example, eyewear can be a pair of eyeglasses. In an example, there can be wrist bands with motion sensors on both (right and left) of a person's wrists to capture eating activity by both the person's dominant and non-dominant hands. In an example, eating-related motions by either hand can trigger activation of the camera on the eyewear. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

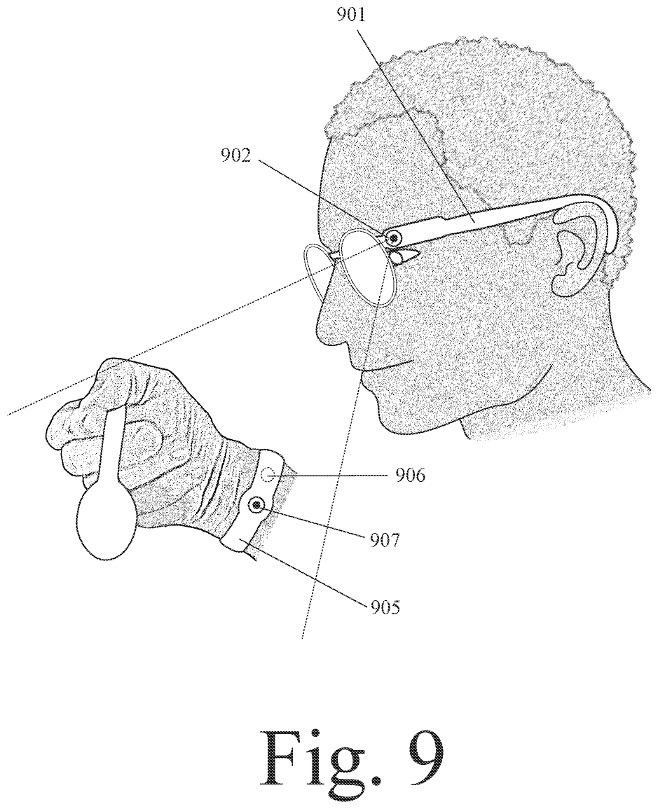

FIG. 9 shows an example of a wearable system for measuring food consumption comprising: an eyewear frame 901 worn by a person; a smart watch (or wrist band) 905 worn by the person; a first camera 902 on the eyewear frame which records food images when activated; a second camera 907 on the smart watch (or wrist band) which records food images when activated; and a motion sensor 906 (e.g. accelerometer and/or gyroscope) on the smart watch (or wrist band), wherein the first camera and/or the second camera are activated to record food images when data from the motion sensor indicates that the person is eating. In an example, eyewear can be a pair of eyeglasses. In an example, there can be wrist bands with motion sensors on both (right and left) of a person's wrists to capture eating activity by both the person's dominant and non-dominant hands. In an example, eating-related motions by either hand can trigger activation of the camera on the eyewear. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the first camera can be part of (or attached to) a sidepiece (e.g. “temple”) of the eyewear frame. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of the eyewear, one directed forward and downward (toward a person's mouth) and one directed straight forward (toward the person's hands). In an example, the focal direction of a camera on eyewear can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

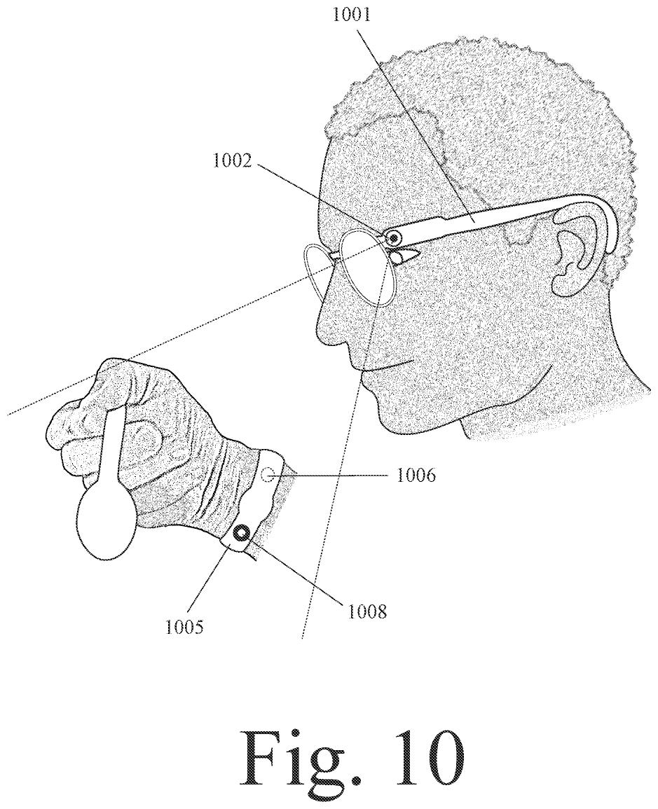

FIG. 10 shows an example of a wearable system for measuring food consumption comprising: an eyewear frame 1001 worn by a person; a camera 1002 on the eyewear frame which records food images when activated; a smart watch (or wrist band) 1005 worn by the person; a motion sensor 1006 (e.g. accelerometer and/or gyroscope) on the smart watch (or wrist band), wherein the camera is activated to record food images when data from the motion sensor indicates that the person is eating; and a spectroscopic sensor 1008 on the smart watch (or wrist band) which analyzes the molecular and/or nutritional composition of food. In an example, a spectroscopic sensor can be activated automatically when data from the motion sensor indicates that the person is eating. In an example, the person can be prompted to use a spectroscopic sensor when data from the motion sensor indicates that the person is eating. In an example, a person can take a spectroscopic scan of food by waving their hand over food. In an example, a spectroscopic sensor can emit light away from the outer surface of a smart watch (or wrist band) and toward food. In an example, a spectroscopic sensor can emit and receive near-infrared light. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, the focal direction of a camera can be changed automatically to track a person's hands. In an example, an indicator light can be on when the camera is activated. In an example, a shutter or flap can automatically cover the camera when the camera is not activated. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands).

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

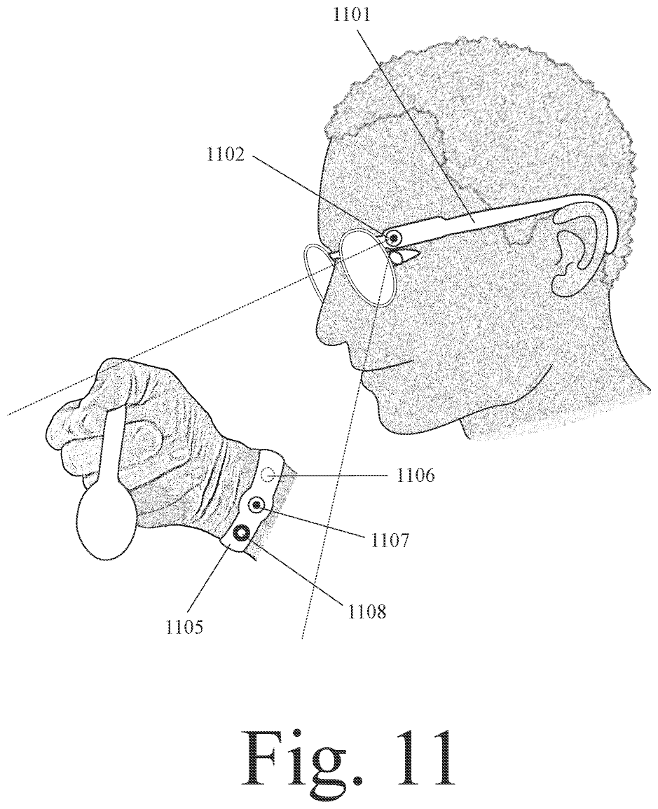

FIG. 11 shows an example of a wearable system for measuring food consumption comprising: an eyewear frame 1101 worn by a person; a smart watch (or wrist band) 1105 worn by the person; a first camera 1102 on the eyewear frame which records food images when activated; a second camera 1107 on the smart watch (or wrist band) which records food images when activated; a motion sensor 1106 (e.g. accelerometer and/or gyroscope) on the smart watch (or wrist band), wherein the first camera and/or the second camera are activated to record food images when data from the motion sensor indicates that the person is eating; and a spectroscopic sensor 1108 on the smart watch (or wrist band) which analyzes the molecular and/or nutritional composition of food. In an example, a spectroscopic sensor can be activated automatically when data from the motion sensor indicates that the person is eating. In an example, the person can be prompted to use a spectroscopic sensor when data from the other sensor(s) indicate that the person is eating. In an example, a person can take a spectroscopic scan of food by waving their hand over food. In an example, a spectroscopic sensor can emit light away from the outer surface of a smart watch (or wrist band) and toward food. In an example, a spectroscopic sensor can emit and receive near-infrared light. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the first camera can be part of (or attached to) a sidepiece (e.g. “temple”) of the eyewear frame. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of the eyewear, one directed forward and downward (toward a person's mouth) and one directed straight forward (toward the person's hands). In an example, the focal direction of a camera on eyewear can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

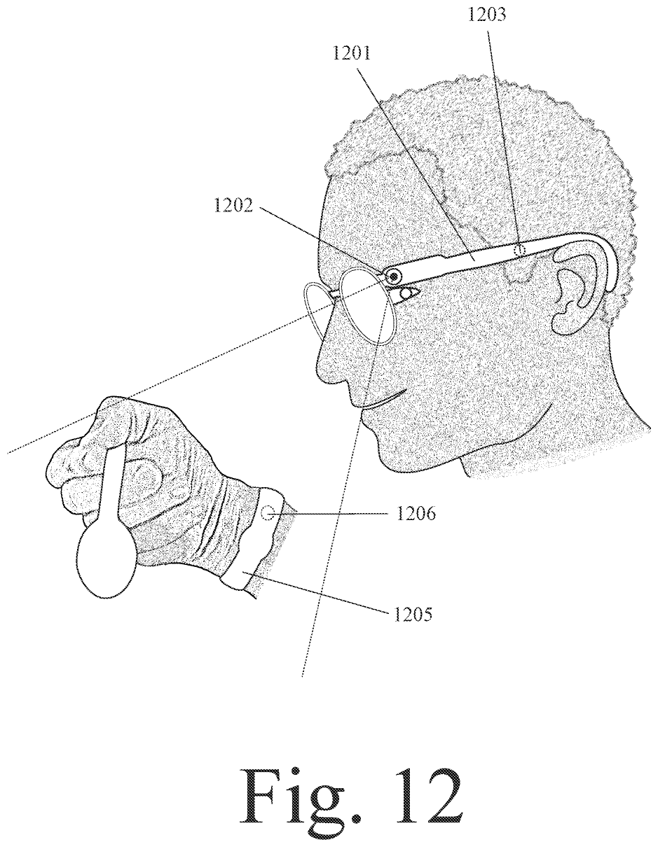

FIG. 12 shows an example of a wearable system for measuring food consumption comprising: an eyewear frame 1201 worn by a person; a camera 1202 on the eyewear frame which records food images when activated; a chewing sensor 1203 on the eyewear frame which detects when the person eats; a smart watch (or wrist band) 1205 worn by the person; and a motion sensor 1206 (e.g. accelerometer and/or gyroscope) on the smart watch (or wrist band), wherein the camera is activated to record food images when data from the chewing sensor and/or data from the motion sensor indicate that the person is eating. In an example, joint analysis of data from the chewing sensor and data from the motion sensor can provide more accurate detection of eating than data from either sensor alone or separate analysis of data from both sensors. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, a chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

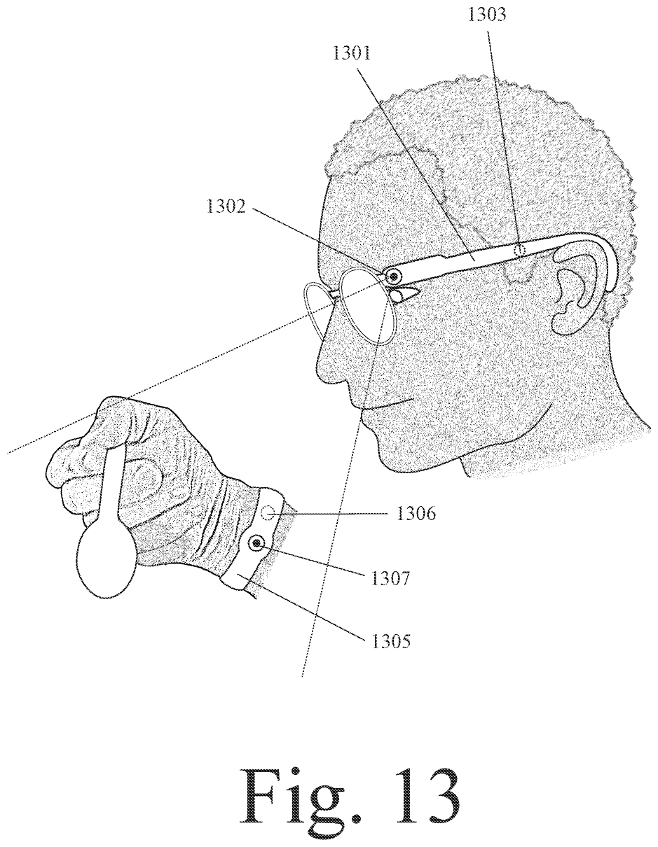

FIG. 13 shows an example of a wearable system for measuring food consumption comprising: an eyewear frame 1301 worn by a person; a chewing sensor 1303 on the eyewear frame which detects when the person eats; a smart watch (or wrist band) 1305 worn by the person; a motion sensor 1306 (e.g. accelerometer and/or gyroscope) on the smart watch (or wrist band); a first camera 1302 on the eyewear frame which records food images when activated, wherein the first camera is activated to record food images when data from the chewing sensor and/or data from the motion sensor indicate that the person is eating; and a second camera 1307 on the smart watch (or wrist band) which records food images when activated, wherein the second camera is activated to record food images when data from the chewing sensor and/or data from the motion sensor indicate that the person is eating. In an example, joint analysis of data from the chewing sensor and data from the motion sensor can provide more accurate detection of eating than data from either sensor alone or separate analysis of data from both sensors. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the first camera can be an integral part of a sidepiece (e.g. “temple”) of smart eyewear. In an example, the first camera can be attached to a sidepiece (e.g. “temple”) of a traditional eyewear. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of the first camera can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

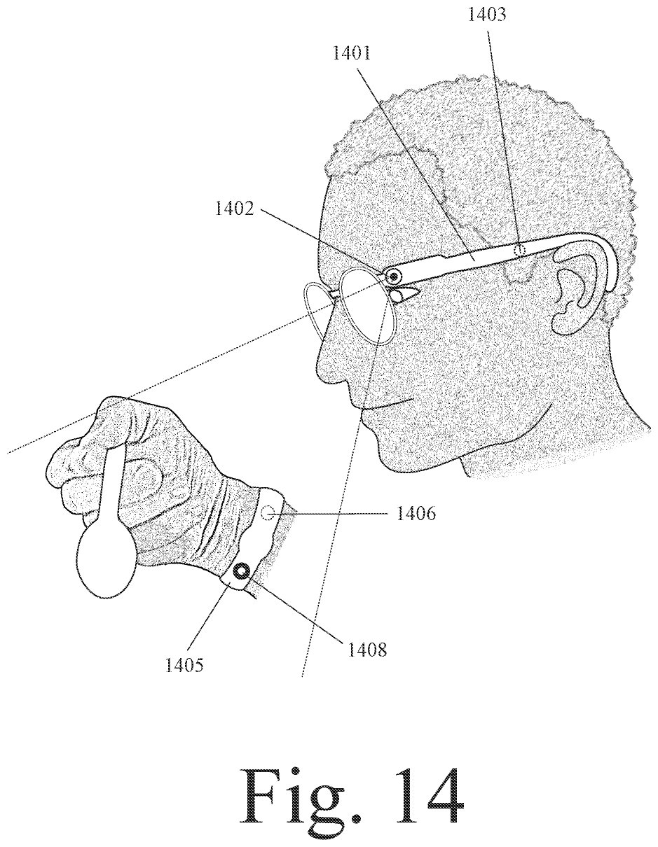

FIG. 14 shows an example of a wearable system for measuring food consumption comprising: an eyewear frame 1401 worn by a person; a chewing sensor 1403 on the eyewear frame which detects when the person eats; a smart watch (or wrist band) 1405 worn by the person; a motion sensor 1406 (e.g. accelerometer and/or gyroscope) on the smart watch (or wrist band) which detects when the person eats; a camera 1402 on the eyewear frame which records food images when activated, wherein the camera is activated to record food images when data from the chewing sensor and/or data from the motion sensor indicate that the person is eating; and a spectroscopic sensor 1408 on the smart watch (or wrist band) which analyzes the molecular and/or nutritional composition of food. In an example, the spectroscopic sensor can be activated automatically when data from the other sensor(s) indicates that the person is eating. In an example, the person can be prompted to use a spectroscopic sensor when data from the other sensor(s) indicate that the person is eating. In an example, a person can take a spectroscopic scan of food by waving their hand over food like Obi-Wan Kenobi (“These aren't the doughnuts you're looking for”). In an example, a spectroscopic sensor can emit light away from the outer surface of a smart watch (or wrist band) and toward food. In an example, a spectroscopic sensor can emit and receive near-infrared light. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor. In an example, a person can take a spectroscopic scan of food by waving their hand over food.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

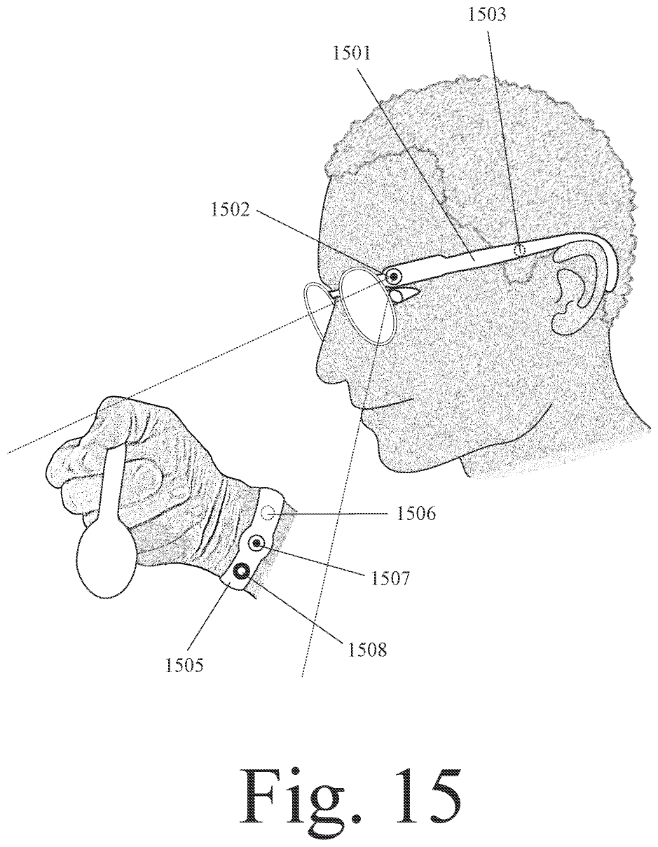

FIG. 15 shows an example of a wearable system for measuring food consumption comprising: an eyewear frame 1501 worn by a person; a chewing sensor 1503 on the eyewear frame which detects when the person eats; a smart watch (or wrist band) 1505 worn by the person; a motion sensor 1506 (e.g. accelerometer and/or gyroscope) on the smart watch (or wrist band) which detects when the person eats; a first camera 1502 on the eyewear frame which records food images when activated, wherein the first camera is activated to record food images when data from the chewing sensor and/or data from the motion sensor indicate that the person is eating; a second camera 1507 on the smart watch (or wrist band) which records food images when activated, wherein the second camera is activated to record food images when data from the chewing sensor and/or data from the motion sensor indicate that the person is eating; and a spectroscopic sensor 1508 on the smart watch (or wrist band) which analyzes the molecular and/or nutritional composition of food. In an example, the spectroscopic sensor can be activated automatically when data from the other sensor(s) indicates that the person is eating. In an example, the person can be prompted to use a spectroscopic sensor when data from the other sensor(s) indicate that the person is eating. In an example, a person can take a spectroscopic scan of food by waving their hand over food. In an example, a spectroscopic sensor can emit light away from the outer surface of a smart watch (or wrist band) and toward food. In an example, a spectroscopic sensor can emit and receive near-infrared light. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the first camera can be an integral part of a sidepiece (e.g. “temple”) of smart eyewear. In an example, the first camera can be attached to a sidepiece (e.g. “temple”) of a traditional eyewear. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of the first camera can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

In an example, a chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

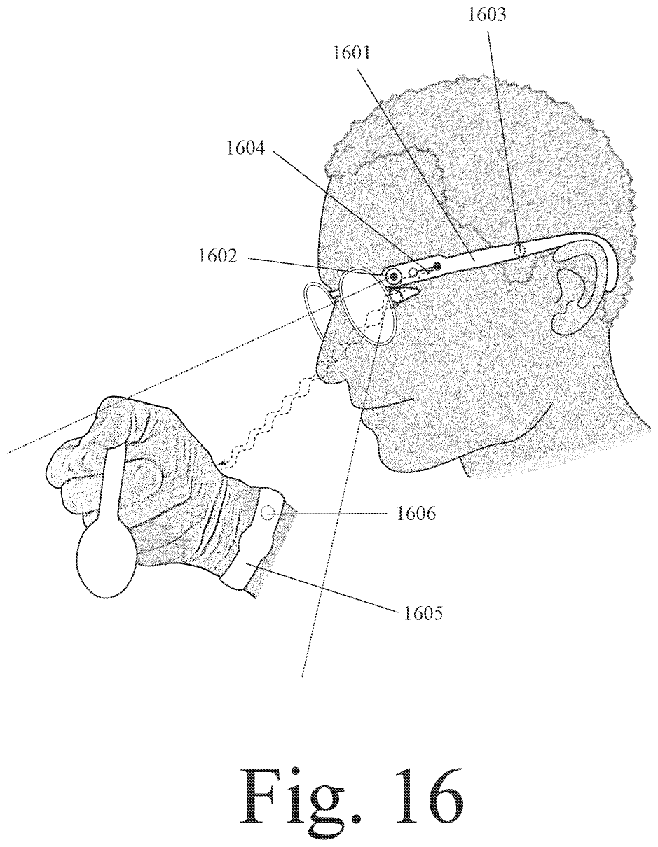

FIG. 16 shows an example of a wearable system for measuring food consumption comprising: an eyewear frame 1601 worn by a person; a chewing sensor 1603 on the eyewear frame which detects when the person eats; a proximity sensor 1604 on the eyewear frame which uses infrared light to detect eating by detecting when an object (such as the person's hand) is near the person's mouth; a smart watch (or wrist band) 1605 worn by the person; a motion sensor 1606 (e.g. accelerometer and/or gyroscope) on the smart watch (or wrist band) which detects when the person eats; and a camera 1602 on the eyewear frame which records food images when activated, wherein the camera is activated to record food images when data from the chewing sensor, data from the proximity sensor, and/or data from the motion sensor indicate that the person is eating. In an example, joint analysis of data from the chewing sensor, the proximity sensor, and the motion sensor can provide more accurate detection of eating than data from any of the three sensors alone or separate analysis of data from the three sensors. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

In an example, the proximity sensor can direct a beam of infrared light toward space in front of the person's mouth. This beam is reflected back toward the proximity sensor when an object (such as the person's hand or a food utensil) is in front of the person's mouth. In an example, the camera can be activated by the proximity sensor to confirm that the person's hand is bringing food up to their mouth, not to brush their teeth, cough, or some other hand-near-mouth gesture.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

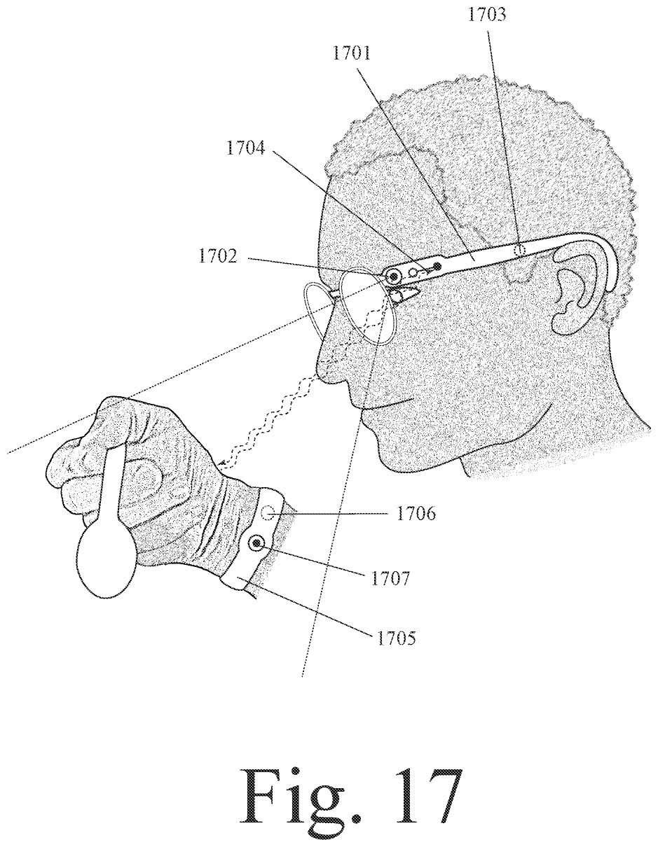

FIG. 17 shows an example of a wearable system for measuring food consumption comprising: an eyewear frame 1701 worn by a person; a chewing sensor 1703 on the eyewear frame which detects when the person eats; a proximity sensor 1704 on the eyewear frame which uses infrared light to detect when the person is eating by detecting when an object (such as the person's hand) is near the person's mouth; a smart watch (or wrist band) 1705 worn by the person; a motion sensor 1706 (e.g. accelerometer and/or gyroscope) on the smart watch (or wrist band); a first camera 1702 on the eyewear frame which records food images when activated, wherein the first camera is activated to record food images when data from the chewing sensor, data from the proximity sensor, and/or data from the motion sensor indicate that the person is eating; and a second camera 1707 on the smart watch (or wrist band) which records food images when activated, wherein the second camera is activated to record food images when data from the chewing sensor, data from the proximity sensor, and/or data from the motion sensor indicate that the person is eating. In an example, joint analysis of data from the chewing sensor, the proximity sensor, and the motion sensor can provide more accurate detection of eating than data from any of the three sensors alone or separate analysis of data from the three sensors. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the first camera can be an integral part of a sidepiece (e.g. “temple”) of smart eyewear. In an example, the first camera can be attached to a sidepiece (e.g. “temple”) of a traditional eyewear. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of the first camera can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

In an example, the proximity sensor can direct a beam of infrared light toward space in front of the person's mouth. This beam is reflected back toward the proximity sensor when an object (such as the person's hand or a food utensil) is in front of the person's mouth. In an example, the camera can be activated by the proximity sensor to confirm that the person's hand is bringing food up to their mouth, not to brush their teeth, cough, or some other hand-near-mouth gesture.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

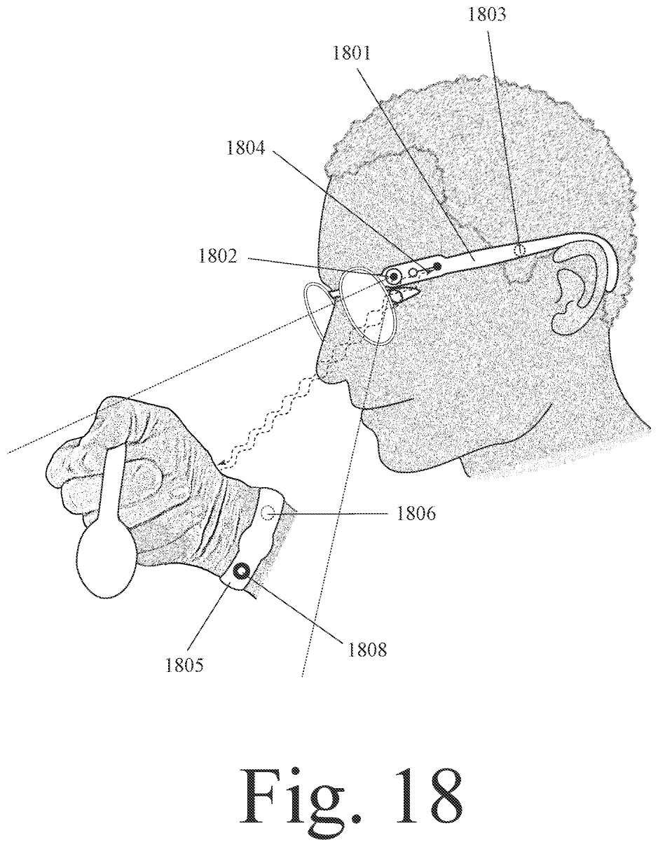

FIG. 18 shows an example of a wearable system for measuring food consumption comprising: an eyewear frame 1801 worn by a person; a chewing sensor 1803 on the eyewear frame which detects when the person eats; a proximity sensor 1804 on the eyewear frame which uses infrared light to detect when the person eats by detecting when an object (such as the person's hand) is near the person's mouth; a smart watch (or wrist band) 1805 worn by the person; a motion sensor 1806 (e.g. accelerometer and/or gyroscope) on the smart watch (or wrist band); a camera 1802 on the eyewear frame which records food images when activated, wherein the camera is activated to record food images when data from the chewing sensor, data from the proximity sensor, and/or data from the motion sensor indicate that the person is eating; and a spectroscopic sensor 1808 on the smart watch (or wrist band) which analyzes the molecular and/or nutritional composition of food. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, joint analysis of data from the chewing sensor, data from the proximity sensor, and data from the motion sensor can provide more accurate detection of eating than data from any of the three sensors alone or separate analysis of data from the three sensors. In an example, the spectroscopic sensor can be activated automatically when data from the other sensor(s) indicates that the person is eating. In an example, a person can be prompted to use a spectroscopic sensor when data from the other sensor(s) indicate that the person is eating. In an example, a person can take a spectroscopic scan of food by waving their hand over food. In an example, a spectroscopic sensor can emit light away from the outer surface of a smart watch (or wrist band) and toward food. In an example, a spectroscopic sensor can emit and receive near-infrared light.

In an example, the camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

In an example, the proximity sensor can direct a beam of infrared light toward space in front of the person's mouth. This beam is reflected back toward the proximity sensor when an object (such as the person's hand or a food utensil) is in front of the person's mouth. In an example, the camera can be activated by the proximity sensor to confirm that the person's hand is bringing food up to their mouth, not to brush their teeth, cough, or some other hand-near-mouth gesture.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

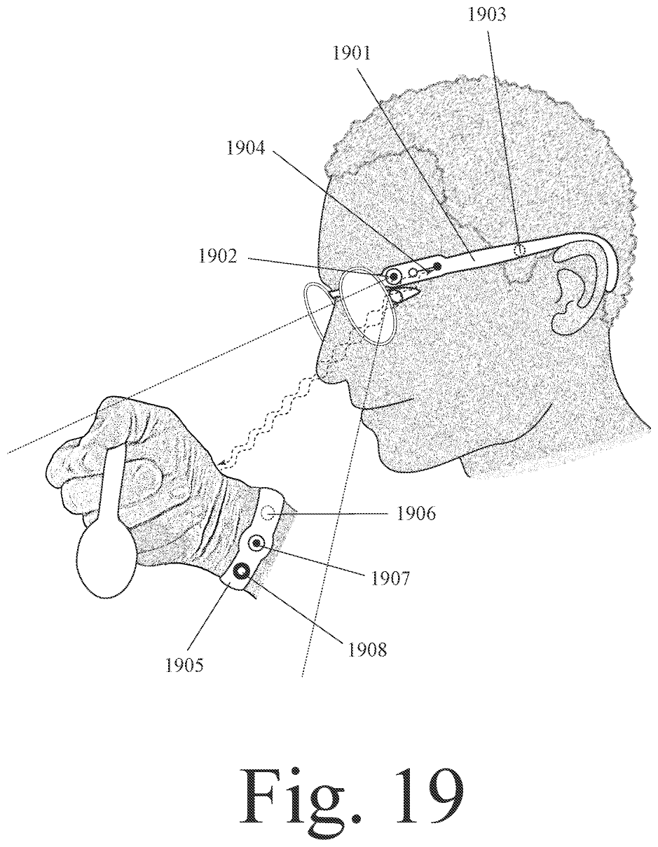

FIG. 19 shows an example of a wearable system for measuring food consumption comprising: an eyewear frame 1901 worn by a person; a chewing sensor 1903 on the eyewear frame which detects when the person eats; a proximity sensor 1904 on the eyewear frame which uses infrared light to detect when the person eats by detecting when an object (such as the person's hand) is near the person's mouth; a smart watch (or wrist band) 1905 worn by the person; a motion sensor 1906 (e.g. accelerometer and/or gyroscope) on the smart watch (or wrist band); a first camera 1902 on the eyewear frame which records food images when activated, wherein the first camera is activated to record food images when data from the chewing sensor, data from the proximity sensor, and/or data from the motion sensor indicate that the person is eating; a second camera 1907 on the smart watch (or wrist band) which records food images when activated, wherein the second camera is activated to record food images when data from the chewing sensor, data from the proximity sensor, and/or data from the motion sensor indicate that the person is eating; and a spectroscopic sensor 1908 on the smart watch (or wrist band) which analyzes the molecular and/or nutritional composition of food. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, joint analysis of data from the chewing sensor, data from the proximity sensor, and data from the motion sensor can provide more accurate detection of eating than data from any of the three sensors alone or separate analysis of data from the three sensors. In an example, the spectroscopic sensor can be activated automatically when data from the other sensor(s) indicates that the person is eating. In an example, a person can be prompted to use a spectroscopic sensor when data from the other sensor(s) indicate that the person is eating. In an example, a person can take a spectroscopic scan of food by waving their hand over food. In an example, a spectroscopic sensor can emit light away from the outer surface of a smart watch (or wrist band) and toward food. In an example, a spectroscopic sensor can emit and receive near-infrared light.

In an example, the first camera can be an integral part of a sidepiece (e.g. “temple”) of smart eyewear. In an example, the first camera can be attached to a sidepiece (e.g. “temple”) of a traditional eyewear. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of the first camera can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

In an example, the proximity sensor can direct a beam of infrared light toward space in front of the person's mouth. This beam is reflected back toward the proximity sensor when an object (such as the person's hand or a food utensil) is in front of the person's mouth. In an example, the camera can be activated by the proximity sensor to confirm that the person's hand is bringing food up to their mouth, not to brush their teeth, cough, or some other hand-near-mouth gesture.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

The following device and system variations can be applied, where relevant, to the examples shown in FIGS. 1 through 19. In an example, a wearable food consumption monitoring system can comprise:

eyeglasses worn by a person; a camera on the eyeglasses; a spectroscopic sensor; and a blood pressure sensor, wherein the camera is triggered to record images and the spectroscopic sensor is activated to make spectroscopic scans when analysis of data from the blood pressure sensor indicates that the person is consuming food. In another example, a wearable food consumption monitoring system can comprise: eyeglasses worn by a person; a camera on the eyeglasses; a spectroscopic sensor; and a piezoelectric sensor, wherein the camera is triggered to record images and the spectroscopic sensor is activated to make spectroscopic scans when analysis of data from the piezoelectric sensor indicates that the person is consuming food. In another example, a wearable food consumption monitoring system can comprise: eyeglasses worn by a person; a camera on the eyeglasses; a spectroscopic sensor; and a swallowing sensor, wherein the camera is triggered to record images and the spectroscopic sensor is activated to make spectroscopic scans when analysis of data from the swallowing sensor indicates that the person is consuming food.

In an example, a wearable food consumption monitoring system can comprise: eyeglasses worn by a person; a camera on the eyeglasses; a spectroscopic sensor; and an optical sensor, wherein the camera is triggered to record images and the spectroscopic sensor is activated to make spectroscopic scans when analysis of data from the optical sensor indicates that the person is consuming food. In another embodiment, a wearable food consumption monitoring system can comprise: eyeglasses worn by a person, wherein the eyeglasses further comprise a camera; a spectroscopic sensor; and a wrist-worn or finger-worn EMG sensor, wherein the camera is triggered to record images and the spectroscopic sensor is activated to make spectroscopic scans when analysis of data from the EMG sensor indicates that the person is consuming food. Alternatively, a wearable food consumption monitoring system can comprise: eyeglasses worn by a person, wherein the eyeglasses further comprise a camera; a spectroscopic sensor; and a wrist-worn or finger-worn optical sensor, wherein the camera is triggered to record images and the spectroscopic sensor is activated to make spectroscopic scans when analysis of data from the optical sensor indicates that the person is consuming food.

In another embodiment, a wearable food consumption monitoring system can comprise: eyeglasses worn by a person, wherein the eyeglasses further comprise a camera; a spectroscopic sensor; and a wrist-worn or finger-worn strain gauge, wherein the camera is triggered to record images and the spectroscopic sensor is activated to make spectroscopic scans when analysis of data from the strain gauge indicates that the person is consuming food. In an example, a wearable food consumption monitoring device can comprise: eyeglasses worn by a person; an infrared sensor on a sidepiece (e.g. a temple) of the eyeglasses, wherein the infrared sensor points toward the person's mouth; at least one EMG sensor on the eyeglasses; and a camera on a sidepiece (e.g. a temple) of the eyeglasses, wherein the camera points toward the person's mouth, and wherein the camera is activated to record food images when analysis of data from the infrared sensor and the at least one EMG sensor indicates that the person is probably eating. In another example, a wearable food consumption monitoring device can comprise: eyeglasses worn by a person; an infrared sensor on the eyeglasses, wherein the infrared sensor points toward the person's mouth; and a camera on a frontpiece and/or nose bridge of the eyeglasses, wherein the camera points toward the person's mouth, and wherein the camera is activated to record food images when analysis of data from the infrared sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; a first camera on a frontpiece and/or nose bridge of the eyeglasses, wherein the first camera points toward the person's mouth; and second camera on a frontpiece and/or nose bridge of the eyeglasses, wherein the second camera points toward the person's mouth, and wherein the first and second cameras are activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor and the at least one EMG sensor indicates that the person is probably eating. In another example, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on the eyeglasses, wherein the camera points toward the person's mouth, and wherein the camera is activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one EMG sensor on the eyeglasses; and a camera on a frontpiece and/or nose bridge of the eyeglasses, wherein the camera points toward the person's mouth, and wherein the camera is activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor and the at least one inertial motion sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one vibration sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor and the at least one vibration sensor indicates that the person is probably eating.

In an example, a wearable food consumption monitoring system can comprise: eyeglasses worn by a person, wherein the eyeglasses further comprise a camera; and a finger-worn motion sensor (e.g. in a smart ring), wherein the camera is triggered to record images along an imaging vector which points toward the person's mouth when analysis of data from the finger-worn motion sensor indicates that the person is consuming food. In another embodiment, a wearable food consumption monitoring system can comprise: eyeglasses worn by a person, wherein the eyeglasses further comprise a camera; and a wrist-worn motion sensor (e.g. in a smart watch), wherein the camera is triggered to record images along an imaging vector which points toward the person's mouth when analysis of data from the wrist-worn motion sensor indicates that the person is consuming food.

In an example, a wearable food consumption monitoring device can comprise: eyeglasses worn by a person, wherein the eyeglasses further comprise a camera; and wherein the eyeglasses further comprise a blood pressure sensor, wherein the camera is triggered to record images along an imaging vector which points toward the person's mouth when analysis of data from the blood pressure sensor indicates that the person is consuming food. In another example, a wearable food consumption monitoring device can comprise: eyeglasses worn by a person, wherein the eyeglasses further comprise a camera; and wherein the eyeglasses further comprise a chewing sensor, wherein a first camera is triggered to record images along an imaging vector which points toward the person's mouth and a second camera is triggered to record images along an imaging vector which points toward a reachable food source when analysis of data from the chewing sensor indicates that the person is consuming food.

In an example, a wearable food consumption monitoring device can comprise: eyeglasses worn by a person, wherein the eyeglasses further comprise a camera; and wherein the eyeglasses further comprise a GPS sensor, wherein a first camera is triggered to record images along an imaging vector which points toward the person's mouth and a second camera is triggered to record images along an imaging vector which points toward a reachable food source when analysis of data from the GPS sensor indicates that the person is consuming food. In an example, a wearable food consumption monitoring device can comprise: eyeglasses worn by a person, wherein the eyeglasses further comprise a camera; and wherein the eyeglasses further comprise a location sensor, wherein the camera is triggered to record images along an imaging vector which points toward the person's mouth when analysis of data from the location sensor indicates that the person is consuming food. In another embodiment, a wearable food consumption monitoring device can comprise: eyeglasses worn by a person, wherein the eyeglasses further comprise a camera; and wherein the eyeglasses further comprise a motion sensor, wherein the camera is triggered to record images along an imaging vector which points toward the person's mouth when analysis of data from the motion sensor indicates that the person is consuming food. In another example, a wearable food consumption monitoring device can comprise: eyeglasses worn by a person, wherein the eyeglasses further comprise a camera; and wherein the eyeglasses further comprise a piezoelectric sensor, wherein a first camera is triggered to record images along an imaging vector which points toward the person's mouth and a second camera is triggered to record images along an imaging vector which points toward a reachable food source when analysis of data from the piezoelectric sensor indicates that the person is consuming food.

In an example, a wearable food consumption monitoring device can comprise: eyeglasses worn by a person, wherein the eyeglasses further comprise a camera; and wherein the eyeglasses further comprise a proximity sensor, wherein a first camera is triggered to record images along an imaging vector which points toward the person's mouth and a second camera is triggered to record images along an imaging vector which points toward a reachable food source when analysis of data from the proximity sensor indicates that the person is consuming food. In another embodiment, a wearable food consumption monitoring device can comprise: eyeglasses worn by a person, wherein the eyeglasses further comprise a camera; and wherein the eyeglasses further comprise a smell sensor, wherein a first camera is triggered to record images along an imaging vector which points toward the person's mouth and a second camera is triggered to record images along an imaging vector which points toward a reachable food source when analysis of data from the smell sensor indicates that the person is consuming food. In another example, a wearable food consumption monitoring device can comprise: eyeglasses worn by a person, wherein the eyeglasses further comprise a camera; and wherein the eyeglasses further comprise a strain gauge, wherein a first camera is triggered to record images along an imaging vector which points toward the person's mouth and a second camera is triggered to record images along an imaging vector which points toward a reachable food source when analysis of data from the strain gauge indicates that the person is consuming food.

In an example, a wearable food consumption monitoring device can comprise: eyeglasses worn by a person, wherein the eyeglasses further comprise a camera; and wherein the eyeglasses further comprise a swallowing sensor, wherein a first camera is triggered to record images along an imaging vector which points toward the person's mouth and a second camera is triggered to record images along an imaging vector which points toward a reachable food source when analysis of data from the swallowing sensor indicates that the person is consuming food. In another embodiment, a wearable food consumption monitoring device can comprise: eyeglasses worn by a person, wherein the eyeglasses further comprise a camera; and wherein the eyeglasses further comprise an EEG sensor, wherein a first camera is triggered to record images along an imaging vector which points toward the person's mouth and a second camera is triggered to record images along an imaging vector which points toward a reachable food source when analysis of data from the EEG sensor indicates that the person is consuming food. Alternatively, a wearable food consumption monitoring device can comprise: eyeglasses worn by a person, wherein the eyeglasses further comprise a camera; and wherein the eyeglasses further comprise an electrochemical sensor, wherein the camera is triggered to record images along an imaging vector which points toward the person's mouth when analysis of data from the electrochemical sensor indicates that the person is consuming food.

In an example, a wearable food consumption monitoring device can comprise: eyeglasses worn by a person, wherein the eyeglasses further comprise a camera; and wherein the eyeglasses further comprise an EMG sensor, wherein the camera is triggered to record images along an imaging vector which points toward the person's mouth when analysis of data from the EMG sensor indicates that the person is consuming food. In another example, a wearable food consumption monitoring device can comprise: eyeglasses worn by a person, wherein the eyeglasses further comprise a camera; and wherein the eyeglasses further comprise an infrared sensor, wherein a first camera is triggered to record images along an imaging vector which points toward the person's mouth and a second camera is triggered to record images along an imaging vector which points toward a reachable food source when analysis of data from the infrared sensor indicates that the person is consuming food. In another embodiment, a wearable food consumption monitoring system can comprise: eyeglasses worn by a person, wherein the eyeglasses further comprise at least two cameras; and wrist-worn motion sensor, wherein a first camera is triggered to record images along an imaging vector which points toward the person's mouth and a second camera is triggered to record images of a reachable food source when analysis of data from the wrist-worn motion sensor indicates that the person is consuming food.

In an example, a wearable food consumption monitoring device can comprise: eyeglasses worn by a person; at least one EMG sensor on a portion of the eyeglasses which curves around the rear of the person's ear; and a camera on a frontpiece and/or nose bridge of the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one EMG sensor indicates that the person is probably eating. In another embodiment, a wearable food consumption monitoring device can comprise: eyeglasses worn by a person; at least one EMG sensor on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one EMG sensor indicates that the person is probably eating.

In an example, a wearable food consumption monitoring device can comprise: eyeglasses worn by a person; at least one EMG sensor on the eyeglasses; and a camera on a sidepiece (e.g. a temple) of the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one inertial motion sensor (e.g. gyroscope and/or accelerometer) on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one inertial motion sensor indicates that the person is probably eating.

In another embodiment, a wearable food consumption monitoring device can comprise: eyeglasses worn by a person; at least one vibration sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one vibration sensor indicates that the person is probably eating. In an example, a wearable food consumption monitoring device can comprise: eyeglasses worn by a person; at least one vibration sensor on the eyeglasses; and a camera on the eyeglasses, wherein the camera points toward the person's mouth, and wherein the camera is activated to record food images when analysis of data from the at least one vibration sensor indicates that the person is probably eating.

In another embodiment, a wearable food consumption monitoring system can comprise: eyeglasses worn by a person; at least one wrist-worn or finger-worn inertial motion sensor (e.g. gyroscope and/or accelerometer on a smart watch or smart ring); and a camera on a frontpiece and/or nose bridge of the eyeglasses, wherein the camera points toward the person's mouth, and wherein the camera is activated to record food images when analysis of data from the at least one wrist-worn or finger-worn inertial motion sensor indicates that the person is probably eating. In an example, a wearable food consumption monitoring device can comprise: eyeglasses worn by a person, wherein the eyeglasses further comprise a camera; wherein the eyeglasses further comprise a motion sensor; and wherein the eyeglasses further comprise an infrared sensor which tracks the location of the person's hands, wherein the camera is triggered to record images along an imaging vector which points toward the person's mouth when joint analysis of data from the motion sensor and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise a chewing sensor, wherein a first camera is triggered to record images along an imaging vector which points toward the person's mouth and a second camera is triggered to record images of a reachable food source when analysis of data from the chewing sensor indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone) and an EEG sensor, wherein a first camera is triggered to record images along an imaging vector which points toward the person's mouth and a second camera is triggered to record images of a reachable food source when joint analysis of data from the sound sensor (e.g. microphone) and the EEG sensor indicates that the person is consuming food. In another embodiment, a wearable food consumption monitoring device can comprise: eyeglasses worn by a person; wherein the eyeglasses further comprise at least two cameras; and wherein the eyeglasses further comprise a sound sensor (e.g. microphone), an EEG sensor, and an infrared sensor, wherein a first camera is triggered to record images along an imaging vector which points toward the person's mouth and a second camera is triggered to record images of a reachable food source when joint analysis of data from the sound sensor (e.g. microphone), the EEG sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone), a chewing sensor, and an infrared sensor, wherein a first camera is triggered to record images along an imaging vector which points toward the person's mouth and a second camera is triggered to record images of a reachable food source when joint analysis of data from the sound sensor (e.g. microphone), the chewing sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a sound sensor (e.g. microphone) and a motion sensor, wherein a first camera is triggered to record images along an imaging vector which points toward the person's mouth and a second camera is triggered to record images of a reachable food source when joint analysis of data from the sound sensor (e.g. microphone) and the motion sensor indicates that the person is consuming food. In an example, a wearable food consumption monitoring device can comprise: eyeglasses worn by a person; wherein the eyeglasses further comprise at least two cameras; and wherein the eyeglasses further comprise a swallow sensor and an EMG sensor, wherein a first camera is triggered to record images along an imaging vector which points toward the person's mouth and a second camera is triggered to record images of a reachable food source when joint analysis of data from the swallow sensor and the EMG sensor indicates that the person is consuming food.

In another embodiment, a wearable food consumption monitoring device can comprise: eyeglasses worn by a person; wherein the eyeglasses further comprise at least two cameras; and wherein the eyeglasses further comprise a swallow sensor, a motion sensor, and an infrared sensor, wherein a first camera is triggered to record images along an imaging vector which points toward the person's mouth and a second camera is triggered to record images of a reachable food source when joint analysis of data from the swallow sensor, the motion sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a swallow sensor, wherein a first camera is triggered to record images along an imaging vector which points toward the person's mouth and a second camera is triggered to record images of a reachable food source when analysis of data from the swallow sensor indicates that the person is consuming food. Alternatively, a wearable food consumption monitoring device can comprise: eyeglasses worn by a person; wherein the eyeglasses further comprise at least two cameras; and wherein the eyeglasses further comprise a swallowing sensor and a chewing sensor, wherein a first camera is triggered to record images along an imaging vector which points toward the person's mouth and a second camera is triggered to record images of a reachable food source when joint analysis of data from the swallowing sensor and the chewing sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor, an EMG sensor, and an infrared sensor, wherein a first camera is triggered to record images along an imaging vector which points toward the person's mouth and a second camera is triggered to record images of a reachable food source when joint analysis of data from the swallowing sensor, the EMG sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise an accelerometer and a chewing sensor, wherein a first camera is triggered to record images along an imaging vector which points toward the person's mouth and a second camera is triggered to record images of a reachable food source when joint analysis of data from the accelerometer and the chewing sensor indicates that the person is consuming food. In an example, a wearable food consumption monitoring device can comprise: eyeglasses worn by a person; wherein the eyeglasses further comprise at least two cameras; and wherein the eyeglasses further comprise an EEG sensor and an accelerometer, wherein a first camera is triggered to record images along an imaging vector which points toward the person's mouth and a second camera is triggered to record images of a reachable food source when joint analysis of data from the EEG sensor and the accelerometer indicates that the person is consuming food.

In another embodiment, a wearable food consumption monitoring device can comprise: eyeglasses worn by a person; wherein the eyeglasses further comprise at least two cameras; and wherein the eyeglasses further comprise an EEG sensor and an accelerometer, wherein a first camera is triggered to record images along an imaging vector which points toward the person's mouth and a second camera is triggered to record images of a reachable food source when joint analysis of data from the EEG sensor and the accelerometer indicates that the person is consuming food. In an example, a wearable food consumption monitoring device can comprise: eyeglasses worn by a person; wherein the eyeglasses further comprise at least two cameras; and wherein the eyeglasses further comprise an EEG sensor, wherein a first camera is triggered to record images along an imaging vector which points toward the person's mouth and a second camera is triggered to record images of a reachable food source when analysis of data from the EEG sensor indicates that the person is consuming food. In another example, a wearable food consumption monitoring device can comprise: eyeglasses worn by a person; wherein the eyeglasses further comprise at least two cameras; and wherein the eyeglasses further comprise an EMG sensor, a sound sensor (e.g. microphone), and an infrared sensor, wherein a first camera is triggered to record images along an imaging vector which points toward the person's mouth and a second camera is triggered to record images of a reachable food source when joint analysis of data from the EMG sensor, the sound sensor (e.g. microphone), and the infrared sensor indicates that the person is consuming food.

In another embodiment, a wearable food consumption monitoring device can comprise: eyeglasses worn by a person; wherein the eyeglasses further comprise at least two cameras; and wherein the eyeglasses further comprise an EMG sensor and a sound sensor (e.g. microphone), wherein a first camera is triggered to record images along an imaging vector which points toward the person's mouth and a second camera is triggered to record images of a reachable food source when joint analysis of data from the EMG sensor and the sound sensor (e.g. microphone) indicates that the person is consuming food. In an example, a wearable food consumption monitoring device can comprise: eyeglasses worn by a person; wherein the eyeglasses further comprise at least two cameras; and wherein the eyeglasses further comprise an motion sensor and an infrared sensor, wherein a first camera is triggered to record images along an imaging vector which points toward the person's mouth and a second camera is triggered to record images of a reachable food source when joint analysis of data from the motion sensor and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a chewing sensor and an infrared sensor, wherein at least one camera is triggered to record images along an imaging vector which points toward the person's mouth when joint analysis of data from the chewing sensor and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a sound sensor (e.g. microphone) and an EEG sensor, wherein at least one camera is triggered to record images along an imaging vector which points toward the person's mouth when joint analysis of data from the sound sensor (e.g. microphone) and the EEG sensor indicates that the person is consuming food. Alternatively, a wearable food consumption monitoring device can comprise: eyeglasses worn by a person; wherein the eyeglasses further comprise one or more cameras; and wherein the eyeglasses further comprise a sound sensor (e.g. microphone), an EEG sensor, and an infrared sensor, wherein at least one camera is triggered to record images along an imaging vector which points toward the person's mouth when joint analysis of data from the sound sensor (e.g. microphone), the EEG sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone) and an EEG sensor, wherein at least one camera is triggered to record images along an imaging vector which points toward the person's mouth when joint analysis of data from the sound sensor (e.g. microphone) and the EEG sensor indicates that the person is consuming food.

In an example, a wearable food consumption monitoring device can comprise: eyeglasses worn by a person; wherein the eyeglasses further comprise one or more cameras; and wherein the eyeglasses further comprise a sound sensor (e.g. microphone), a motion sensor, and an infrared sensor, wherein at least one camera is triggered to record images along an imaging vector which points toward the person's mouth when joint analysis of data from the sound sensor (e.g. microphone), the motion sensor, and the infrared sensor indicates that the person is consuming food. In another embodiment, a wearable food consumption monitoring device can comprise: eyeglasses worn by a person; wherein the eyeglasses further comprise one or more cameras; and wherein the eyeglasses further comprise a swallow sensor and an EMG sensor, wherein at least one camera is triggered to record images along an imaging vector which points toward the person's mouth when joint analysis of data from the swallow sensor and the EMG sensor indicates that the person is consuming food. In an example, a wearable food consumption monitoring device can comprise: eyeglasses worn by a person; wherein the eyeglasses further comprise one or more cameras; and wherein the eyeglasses further comprise a swallow sensor, a motion sensor, and an infrared sensor, wherein at least one camera is triggered to record images along an imaging vector which points toward the person's mouth when joint analysis of data from the swallow sensor, the motion sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallow sensor, wherein at least one camera is triggered to record images along an imaging vector which points toward the person's mouth when analysis of data from the swallow sensor indicates that the person is consuming food. In another example, a wearable food consumption monitoring device can comprise: eyeglasses worn by a person; wherein the eyeglasses further comprise one or more cameras; and wherein the eyeglasses further comprise a swallowing sensor and a chewing sensor, wherein at least one camera is triggered to record images along an imaging vector which points toward the person's mouth when joint analysis of data from the swallowing sensor and the chewing sensor indicates that the person is consuming food.

In an example, a wearable food consumption monitoring device can comprise: eyeglasses worn by a person; wherein the eyeglasses further comprise one or more cameras; and wherein the eyeglasses further comprise a swallowing sensor, an accelerometer, and an infrared sensor, wherein at least one camera is triggered to record images along an imaging vector which points toward the person's mouth when joint analysis of data from the swallowing sensor, the accelerometer, and the infrared sensor indicates that the person is consuming food. In another embodiment, a wearable food consumption monitoring device can comprise: eyeglasses worn by a person; wherein the eyeglasses further comprise one or more cameras; and wherein the eyeglasses further comprise an accelerometer and a chewing sensor, wherein at least one camera is triggered to record images along an imaging vector which points toward the person's mouth when joint analysis of data from the accelerometer and the chewing sensor indicates that the person is consuming food. In another example, a wearable food consumption monitoring device can comprise: eyeglasses worn by a person; wherein the eyeglasses further comprise one or more cameras; and wherein the eyeglasses further comprise an EEG sensor and an accelerometer, wherein at least one camera is triggered to record images along an imaging vector which points toward the person's mouth when joint analysis of data from the EEG sensor and the accelerometer indicates that the person is consuming food.

In an example, a wearable food consumption monitoring device can comprise: eyeglasses worn by a person; wherein the eyeglasses further comprise one or more cameras; and wherein the eyeglasses further comprise an EEG sensor and an accelerometer, wherein at least one camera is triggered to record images along an imaging vector which points toward the person's mouth when joint analysis of data from the EEG sensor and the accelerometer indicates that the person is consuming food. In another embodiment, a wearable food consumption monitoring device can comprise: eyeglasses worn by a person; wherein the eyeglasses further comprise one or more cameras; and wherein the eyeglasses further comprise an EEG sensor, wherein at least one camera is triggered to record images along an imaging vector which points toward the person's mouth when analysis of data from the EEG sensor indicates that the person is consuming food.

In an example, a wearable food consumption monitoring device can comprise: eyeglasses worn by a person; wherein the eyeglasses further comprise one or more cameras; and wherein the eyeglasses further comprise an EMG sensor and a sound sensor (e.g. microphone), wherein at least one camera is triggered to record images along an imaging vector which points toward the person's mouth when joint analysis of data from the EMG sensor and the sound sensor (e.g. microphone) indicates that the person is consuming food. In another example, a wearable food consumption monitoring device can comprise: eyeglasses worn by a person; wherein the eyeglasses further comprise one or more cameras; and wherein the eyeglasses further comprise an EMG sensor, a motion sensor, and an infrared sensor, wherein at least one camera is triggered to record images along an imaging vector which points toward the person's mouth when joint analysis of data from the EMG sensor, the motion sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise an EMG sensor, wherein at least one camera is triggered to record images along an imaging vector which points toward the person's mouth when analysis of data from the EMG sensor indicates that the person is consuming food. In an example, a wearable food consumption monitoring device can comprise: eyeglasses worn by a person; wherein the eyeglasses further comprise one or more cameras; and wherein the eyeglasses further comprise an motion sensor, a chewing sensor, and an infrared sensor, wherein at least one camera is triggered to record images along an imaging vector which points toward the person's mouth when joint analysis of data from the motion sensor, the chewing sensor, and the infrared sensor indicates that the person is consuming food. In another embodiment, a wearable food consumption monitoring system can comprise: eyeglasses worn by a person; a camera on the eyeglasses; a spectroscopic sensor; and a chewing sensor, wherein the camera is triggered to record images and the spectroscopic sensor is activated to make spectroscopic scans when analysis of data from the chewing sensor indicates that the person is consuming food.

In an example, a wearable food consumption monitoring system can comprise: eyeglasses worn by a person; a camera on the eyeglasses; a spectroscopic sensor; and a pressure sensor, wherein the camera is triggered to record images and the spectroscopic sensor is activated to make spectroscopic scans when analysis of data from the pressure sensor indicates that the person is consuming food. In another example, a wearable food consumption monitoring system can comprise: eyeglasses worn by a person; a camera on the eyeglasses; a spectroscopic sensor; and an EEG sensor, wherein the camera is triggered to record images and the spectroscopic sensor is activated to make spectroscopic scans when analysis of data from the EEG sensor indicates that the person is consuming food.

In another embodiment, a wearable food consumption monitoring system can comprise: eyeglasses worn by a person, wherein the eyeglasses further comprise a camera; a spectroscopic sensor; and a wrist-worn or finger-worn blood pressure sensor, wherein the camera is triggered to record images and the spectroscopic sensor is activated to make spectroscopic scans when analysis of data from the blood pressure sensor indicates that the person is consuming food. In another example, a wearable food consumption monitoring system can comprise: eyeglasses worn by a person, wherein the eyeglasses further comprise a camera; a spectroscopic sensor; and a wrist-worn or finger-worn piezoelectric sensor, wherein the camera is triggered to record images and the spectroscopic sensor is activated to make spectroscopic scans when analysis of data from the piezoelectric sensor indicates that the person is consuming food.

In an example, a wearable food consumption monitoring device can comprise: eyeglasses worn by a person; an infrared sensor on a sidepiece (e.g. a temple) of the eyeglasses, wherein the infrared sensor points toward the person's mouth; at least one inertial motion sensor (e.g. gyroscope and/or accelerometer) on the eyeglasses; and a camera on a sidepiece (e.g. a temple) of the eyeglasses, wherein the camera points toward the person's mouth, and wherein the camera is activated to record food images when analysis of data from the infrared sensor and the at least one inertial motion sensor indicates that the person is probably eating. In an example, a wearable food consumption monitoring device can comprise: eyeglasses worn by a person; an infrared sensor on the eyeglasses, wherein the infrared sensor points toward the person's mouth; and a camera on the eyeglasses, wherein the camera points toward the person's mouth, and wherein the camera is activated to record food images when analysis of data from the infrared sensor indicates that the person is probably eating.

In an example, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor and the at least one EMG sensor indicates that the person is probably eating. In another example, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the infrared sensor and the at least one EMG sensor indicates that the person is probably eating. In another embodiment, a wearable food consumption monitoring device can comprise: eyeglasses worn by a person; an infrared sensor on the eyeglasses, wherein the infrared sensor points toward the person's mouth; at least one EMG sensor on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one EMG sensor on the eyeglasses; and a camera on the eyeglasses, wherein the camera points toward the person's mouth, and wherein the camera is activated to record food images when analysis of data from the infrared sensor and the at least one EMG sensor indicates that the person is probably eating. In another example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; and a camera on a frontpiece and/or nose bridge of the eyeglasses, wherein the camera points toward the person's mouth, and wherein the camera is activated to record food images when analysis of data from the infrared sensor and the at least one inertial motion sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one vibration sensor on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor and the at least one vibration sensor indicates that the person is probably eating. Alternatively, a wearable food consumption monitoring system can comprise: eyeglasses worn by a person, wherein the eyeglasses further comprise at least two cameras; and a finger-worn motion sensor, wherein a first camera is triggered to record images along an imaging vector which points toward the person's mouth and a second camera is triggered to record images of a reachable food source when analysis of data from the wrist-worn motion sensor indicates that the person is consuming food.

In another embodiment, a wearable food consumption monitoring system can comprise: eyeglasses worn by a person, wherein the eyeglasses further comprise a camera; and a wrist-worn motion sensor, wherein the camera is triggered to record food images when analysis of data from the wrist-worn motion sensor indicates that the person is consuming food. In another example, a wearable food consumption monitoring device can comprise: eyeglasses worn by a person, wherein the eyeglasses further comprise a camera; and wherein the eyeglasses further comprise a blood pressure sensor, wherein a first camera is triggered to record images along an imaging vector which points toward the person's mouth and a second camera is triggered to record images along an imaging vector which points toward a reachable food source when analysis of data from the blood pressure sensor indicates that the person is consuming food.

In an example, a wearable food consumption monitoring device can comprise: eyeglasses worn by a person, wherein the eyeglasses further comprise a camera; and wherein the eyeglasses further comprise a chewing sensor, wherein the camera is triggered to record images along an imaging vector which points toward the person's mouth when analysis of data from the chewing sensor indicates that the person is consuming food. In another embodiment, a wearable food consumption monitoring device can comprise: eyeglasses worn by a person, wherein the eyeglasses further comprise a camera; and wherein the eyeglasses further comprise a GPS sensor, wherein the camera is triggered to record images along an imaging vector which points toward the person's mouth when analysis of data from the GPS sensor indicates that the person is consuming food. In another example, a wearable food consumption monitoring device can comprise: eyeglasses worn by a person, wherein the eyeglasses further comprise a camera; and wherein the eyeglasses further comprise a microphone, wherein the camera is triggered to record images of the interaction between food and the person's mouth when analysis of data from sensor indicates that the person is consuming food.

In an example, a wearable food consumption monitoring device can comprise: eyeglasses worn by a person, wherein the eyeglasses further comprise a camera; and wherein the eyeglasses further comprise a motion sensor, wherein a first camera is triggered to record images along an imaging vector which points toward the person's mouth and a second camera is triggered to record images along an imaging vector which points toward a reachable food source when analysis of data from the motion sensor indicates that the person is consuming food. In an example, a wearable food consumption monitoring device can comprise: eyeglasses worn by a person, wherein the eyeglasses further comprise a camera; and wherein the eyeglasses further comprise a piezoelectric sensor, wherein the camera is triggered to record images along an imaging vector which points toward the person's mouth when analysis of data from the piezoelectric sensor indicates that the person is consuming food.

In an example, a wearable food consumption monitoring device can comprise: eyeglasses worn by a person, wherein the eyeglasses further comprise a camera; and wherein the eyeglasses further comprise a proximity sensor, wherein the camera is triggered to record images along an imaging vector which points toward the person's mouth when analysis of data from the proximity sensor indicates that the person is consuming food. In an example, a wearable food consumption monitoring device can comprise: eyeglasses worn by a person, wherein the eyeglasses further comprise a camera; and wherein the eyeglasses further comprise a smell sensor, wherein the camera is triggered to record images along an imaging vector which points toward the person's mouth when analysis of data from the smell sensor indicates that the person is consuming food. In an example, a wearable food consumption monitoring device can comprise: eyeglasses worn by a person, wherein the eyeglasses further comprise a camera; and wherein the eyeglasses further comprise a strain gauge, wherein the camera is triggered to record images along an imaging vector which points toward the person's mouth when analysis of data from the strain gauge indicates that the person is consuming food. In an example, a wearable food consumption monitoring device can comprise: eyeglasses worn by a person, wherein the eyeglasses further comprise a camera; and wherein the eyeglasses further comprise a swallowing sensor, wherein the camera is triggered to record images along an imaging vector which points toward the person's mouth when analysis of data from the swallowing sensor indicates that the person is consuming food.

In another embodiment, a wearable food consumption monitoring device can comprise: eyeglasses worn by a person, wherein the eyeglasses further comprise a camera; and wherein the eyeglasses further comprise an EEG sensor, wherein the camera is triggered to record images along an imaging vector which points toward the person's mouth when analysis of data from the EEG sensor indicates that the person is consuming food. In an example, a wearable food consumption monitoring device can comprise: eyeglasses worn by a person, wherein the eyeglasses further comprise a camera; and wherein the eyeglasses further comprise an EMG sensor, wherein a first camera is triggered to record images along an imaging vector which points toward the person's mouth and a second camera is triggered to record images along an imaging vector which points toward a reachable food source when analysis of data from the EMG sensor indicates that the person is consuming food. In another example, a wearable food consumption monitoring device can comprise: eyeglasses worn by a person, wherein the eyeglasses further comprise a camera; and wherein the eyeglasses further comprise an EMG sensor, wherein the camera is triggered to record images of the interaction between food and the person's mouth when analysis of data from sensor indicates that the person is consuming food. Alternatively, a wearable food consumption monitoring device can comprise: eyeglasses worn by a person, wherein the eyeglasses further comprise a camera; and wherein the eyeglasses further comprise an infrared sensor, wherein the camera is triggered to record images along an imaging vector which points toward the person's mouth when analysis of data from the infrared sensor indicates that the person is consuming food.

In an example, a wearable food consumption monitoring device can comprise: eyeglasses worn by a person; at least one EMG sensor on a portion of the eyeglasses which curves around the rear of the person's ear; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one EMG sensor indicates that the person is probably eating. In another example, a wearable food consumption monitoring device can comprise: eyeglasses worn by a person; at least one EMG sensor on a portion of the eyeglasses which curves around the rear of the person's ear; and a camera on a sidepiece (e.g. a temple) of the eyeglasses, wherein the camera points toward the person's mouth, and wherein the camera is activated to record food images when analysis of data from the at least one EMG sensor indicates that the person is probably eating.

In an example, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one EMG sensor indicates that the person is probably eating. In an example, a wearable food consumption monitoring device can comprise: eyeglasses worn by a person; at least one EMG sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one EMG sensor indicates that the person is probably eating.

In another embodiment, a wearable food consumption monitoring device can comprise: eyeglasses worn by a person; at least one EMG sensor on the eyeglasses; and a camera on the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one inertial motion sensor (e.g. gyroscope and/or accelerometer) on the eyeglasses; and a camera on a frontpiece and/or nose bridge of the eyeglasses, wherein the camera points toward the person's mouth, and wherein the camera is activated to record food images when analysis of data from the at least one inertial motion sensor indicates that the person is probably eating. In an example, a wearable food consumption monitoring device can comprise: eyeglasses worn by a person; at least one vibration sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one vibration sensor indicates that the person is probably eating.

In another embodiment, a wearable food consumption monitoring system can comprise: eyeglasses worn by a person; at least one wrist-worn or finger-worn inertial motion sensor (e.g. gyroscope and/or accelerometer on a smart watch or smart ring); a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one wrist-worn or finger-worn inertial motion sensor indicates that the person is probably eating. In an example, a wearable food consumption monitoring system can comprise: eyeglasses worn by a person; at least one wrist-worn or finger-worn inertial motion sensor (e.g. gyroscope and/or accelerometer on a smart watch or smart ring); and a camera on a sidepiece (e.g. a temple) of the eyeglasses, wherein the camera points toward the person's mouth, and wherein the camera is activated to record food images when analysis of data from the at least one wrist-worn or finger-worn inertial motion sensor indicates that the person is probably eating.

In another embodiment, a wearable food consumption monitoring device can comprise: eyeglasses worn by a person, wherein the eyeglasses further comprise a camera; wherein the eyeglasses further comprise an EMG sensor; and wherein the eyeglasses further comprise an infrared sensor which tracks the location of the person's hands, wherein the camera is triggered to record images along an imaging vector which points toward the person's mouth when joint analysis of data from the EMG sensor and the infrared sensor indicates that the person is consuming food. Alternatively, a wearable food consumption monitoring device can comprise: eyeglasses worn by a person; wherein the eyeglasses further comprise at least two cameras; and wherein the eyeglasses further comprise a motion sensor, wherein a first camera is triggered to record images along an imaging vector which points toward the person's mouth and a second camera is triggered to record images of a reachable food source when analysis of data from the motion sensor indicates that the person is consuming food.

In another embodiment, a wearable food consumption monitoring device can comprise: eyeglasses worn by a person; wherein the eyeglasses further comprise at least two cameras; and wherein the eyeglasses further comprise a sound sensor (e.g. microphone) and a motion sensor, wherein a first camera is triggered to record images along an imaging vector which points toward the person's mouth and a second camera is triggered to record images of a reachable food source when joint analysis of data from the sound sensor (e.g. microphone) and the motion sensor indicates that the person is consuming food. In another example, a wearable food consumption monitoring device can comprise: eyeglasses worn by a person; wherein the eyeglasses further comprise at least two cameras; and wherein the eyeglasses further comprise a sound sensor (e.g. microphone), a motion sensor, and an infrared sensor, wherein a first camera is triggered to record images along an imaging vector which points toward the person's mouth and a second camera is triggered to record images of a reachable food source when joint analysis of data from the sound sensor (e.g. microphone), the motion sensor, and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise a sound sensor (e.g. microphone) and an infrared sensor, wherein a first camera is triggered to record images along an imaging vector which points toward the person's mouth and a second camera is triggered to record images of a reachable food source when joint analysis of data from the sound sensor (e.g. microphone) and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone), a motion sensor, and an infrared sensor, wherein a first camera is triggered to record images along an imaging vector which points toward the person's mouth and a second camera is triggered to record images of a reachable food source when joint analysis of data from the sound sensor (e.g. microphone), the motion sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a swallow sensor and a sound sensor (e.g. microphone), wherein a first camera is triggered to record images along an imaging vector which points toward the person's mouth and a second camera is triggered to record images of a reachable food source when joint analysis of data from the swallow sensor and the sound sensor (e.g. microphone) indicates that the person is consuming food. Alternatively, a wearable food consumption monitoring device can comprise: eyeglasses worn by a person; wherein the eyeglasses further comprise at least two cameras; and wherein the eyeglasses further comprise a swallow sensor, a sound sensor (e.g. microphone), and an infrared sensor, wherein a first camera is triggered to record images along an imaging vector which points toward the person's mouth and a second camera is triggered to record images of a reachable food source when joint analysis of data from the swallow sensor, the sound sensor (e.g. microphone), and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor and an infrared sensor, wherein a first camera is triggered to record images along an imaging vector which points toward the person's mouth and a second camera is triggered to record images of a reachable food source when joint analysis of data from the swallowing sensor and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a swallowing sensor and an accelerometer, wherein a first camera is triggered to record images along an imaging vector which points toward the person's mouth and a second camera is triggered to record images of a reachable food source when joint analysis of data from the swallowing sensor and the accelerometer indicates that the person is consuming food. In an example, a wearable food consumption monitoring device can comprise: eyeglasses worn by a person; wherein the eyeglasses further comprise at least two cameras; and wherein the eyeglasses further comprise a swallowing sensor, an accelerometer, and an infrared sensor, wherein a first camera is triggered to record images along an imaging vector which points toward the person's mouth and a second camera is triggered to record images of a reachable food source when joint analysis of data from the swallowing sensor, the accelerometer, and the infrared sensor indicates that the person is consuming food.

In another embodiment, a wearable food consumption monitoring device can comprise: eyeglasses worn by a person; wherein the eyeglasses further comprise at least two cameras; and wherein the eyeglasses further comprise an accelerometer and an infrared sensor, wherein a first camera is triggered to record images along an imaging vector which points toward the person's mouth and a second camera is triggered to record images of a reachable food source when joint analysis of data from the accelerometer and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise an EEG sensor and a chewing sensor, wherein a first camera is triggered to record images along an imaging vector which points toward the person's mouth and a second camera is triggered to record images of a reachable food source when joint analysis of data from the EEG sensor and the chewing sensor indicates that the person is consuming food. Alternatively, a wearable food consumption monitoring device can comprise: eyeglasses worn by a person; wherein the eyeglasses further comprise at least two cameras; and wherein the eyeglasses further comprise an EEG sensor, a chewing sensor, and an infrared sensor, wherein a first camera is triggered to record images along an imaging vector which points toward the person's mouth and a second camera is triggered to record images of a reachable food source when joint analysis of data from the EEG sensor, the chewing sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise an EMG sensor and an accelerometer, wherein a first camera is triggered to record images along an imaging vector which points toward the person's mouth and a second camera is triggered to record images of a reachable food source when joint analysis of data from the EMG sensor and the accelerometer indicates that the person is consuming food. In another example, a wearable food consumption monitoring device can comprise: eyeglasses worn by a person; wherein the eyeglasses further comprise at least two cameras; and wherein the eyeglasses further comprise an EMG sensor and an infrared sensor, wherein a first camera is triggered to record images along an imaging vector which points toward the person's mouth and a second camera is triggered to record images of a reachable food source when joint analysis of data from the EMG sensor and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise an EMG sensor, an EEG sensor, and an infrared sensor, wherein a first camera is triggered to record images along an imaging vector which points toward the person's mouth and a second camera is triggered to record images of a reachable food source when joint analysis of data from the EMG sensor, the EEG sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise an motion sensor and a chewing sensor, wherein a first camera is triggered to record images along an imaging vector which points toward the person's mouth and a second camera is triggered to record images of a reachable food source when joint analysis of data from the motion sensor and the chewing sensor indicates that the person is consuming food. In another example, a wearable food consumption monitoring device can comprise: eyeglasses worn by a person; wherein the eyeglasses further comprise one or more cameras; and wherein the eyeglasses further comprise a chewing sensor, wherein at least one camera is triggered to record images along an imaging vector which points toward the person's mouth when analysis of data from the chewing sensor indicates that the person is consuming food. In an example, a wearable food consumption monitoring device can comprise: eyeglasses worn by a person; wherein the eyeglasses further comprise one or more cameras; and wherein the eyeglasses further comprise a sound sensor (e.g. microphone) and a motion sensor, wherein at least one camera is triggered to record images along an imaging vector which points toward the person's mouth when joint analysis of data from the sound sensor (e.g. microphone) and the motion sensor indicates that the person is consuming food.

In another embodiment, a wearable food consumption monitoring device can comprise: eyeglasses worn by a person; wherein the eyeglasses further comprise one or more cameras; and wherein the eyeglasses further comprise a sound sensor (e.g. microphone), a motion sensor, and an infrared sensor, wherein at least one camera is triggered to record images along an imaging vector which points toward the person's mouth when joint analysis of data from the sound sensor (e.g. microphone), the motion sensor, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise a sound sensor (e.g. microphone) and a chewing sensor, wherein at least one camera is triggered to record images along an imaging vector which points toward the person's mouth when joint analysis of data from the sound sensor (e.g. microphone) and the chewing sensor indicates that the person is consuming food. In another embodiment, a wearable food consumption monitoring device can comprise: eyeglasses worn by a person; wherein the eyeglasses further comprise one or more cameras; and wherein the eyeglasses further comprise a sound sensor (e.g. microphone) and an infrared sensor, wherein at least one camera is triggered to record images along an imaging vector which points toward the person's mouth when joint analysis of data from the sound sensor (e.g. microphone) and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallow sensor and a sound sensor (e.g. microphone), wherein at least one camera is triggered to record images along an imaging vector which points toward the person's mouth when joint analysis of data from the swallow sensor and the sound sensor (e.g. microphone) indicates that the person is consuming food. In another example, a wearable food consumption monitoring device can comprise: eyeglasses worn by a person; wherein the eyeglasses further comprise one or more cameras; and wherein the eyeglasses further comprise a swallow sensor, a sound sensor (e.g. microphone), and an infrared sensor, wherein at least one camera is triggered to record images along an imaging vector which points toward the person's mouth when joint analysis of data from the swallow sensor, the sound sensor (e.g. microphone), and the infrared sensor indicates that the person is consuming food. In another embodiment, a wearable food consumption monitoring device can comprise: eyeglasses worn by a person; wherein the eyeglasses further comprise one or more cameras; and wherein the eyeglasses further comprise a swallowing sensor and an infrared sensor, wherein at least one camera is triggered to record images along an imaging vector which points toward the person's mouth when joint analysis of data from the swallowing sensor and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallowing sensor and an accelerometer, wherein at least one camera is triggered to record images along an imaging vector which points toward the person's mouth when joint analysis of data from the swallowing sensor and the accelerometer indicates that the person is consuming food. In another example, a wearable food consumption monitoring device can comprise: eyeglasses worn by a person; wherein the eyeglasses further comprise one or more cameras; and wherein the eyeglasses further comprise a swallowing sensor, a sound sensor (e.g. microphone), and an infrared sensor, wherein at least one camera is triggered to record images along an imaging vector which points toward the person's mouth when joint analysis of data from the swallowing sensor, the sound sensor (e.g. microphone), and the infrared sensor indicates that the person is consuming food. In another embodiment, a wearable food consumption monitoring device can comprise: eyeglasses worn by a person; wherein the eyeglasses further comprise one or more cameras; and wherein the eyeglasses further comprise an accelerometer and an infrared sensor, wherein at least one camera is triggered to record images along an imaging vector which points toward the person's mouth when joint analysis of data from the accelerometer and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise an EEG sensor and a chewing sensor, wherein at least one camera is triggered to record images along an imaging vector which points toward the person's mouth when joint analysis of data from the EEG sensor and the chewing sensor indicates that the person is consuming food. In another example, a wearable food consumption monitoring device can comprise: eyeglasses worn by a person; wherein the eyeglasses further comprise one or more cameras; and wherein the eyeglasses further comprise an EEG sensor, a chewing sensor, and an infrared sensor, wherein at least one camera is triggered to record images along an imaging vector which points toward the person's mouth when joint analysis of data from the EEG sensor, the chewing sensor, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise an EMG sensor and an accelerometer, wherein at least one camera is triggered to record images along an imaging vector which points toward the person's mouth when joint analysis of data from the EMG sensor and the accelerometer indicates that the person is consuming food.

In an example, a wearable food consumption monitoring device can comprise: eyeglasses worn by a person; wherein the eyeglasses further comprise one or more cameras; and wherein the eyeglasses further comprise an EMG sensor and an infrared sensor, wherein at least one camera is triggered to record images along an imaging vector which points toward the person's mouth when joint analysis of data from the EMG sensor and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an EMG sensor, a sound sensor (e.g. microphone), and an infrared sensor, wherein at least one camera is triggered to record images along an imaging vector which points toward the person's mouth when joint analysis of data from the EMG sensor, the sound sensor (e.g. microphone), and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an motion sensor and a chewing sensor, wherein at least one camera is triggered to record images along an imaging vector which points toward the person's mouth when joint analysis of data from the motion sensor and the chewing sensor indicates that the person is consuming food.

In another embodiment, a wearable food consumption monitoring device can comprise: eyeglasses worn by a person; wherein the eyeglasses further comprise one or more cameras; and wherein the eyeglasses further comprise an motion sensor, wherein at least one camera is triggered to record images along an imaging vector which points toward the person's mouth when analysis of data from the motion sensor indicates that the person is consuming food. In another example, a wearable food consumption monitoring system can comprise: eyeglasses worn by a person; a camera on the eyeglasses; a spectroscopic sensor; and a GPS sensor, wherein the camera is triggered to record images and the spectroscopic sensor is activated to make spectroscopic scans when analysis of data from the GPS sensor indicates that the person is consuming food. In an example, a wearable food consumption monitoring system can comprise: eyeglasses worn by a person; a camera on the eyeglasses; a spectroscopic sensor; and a proximity sensor, wherein the camera is triggered to record images and the spectroscopic sensor is activated to make spectroscopic scans when analysis of data from the proximity sensor indicates that the person is consuming food. Alternatively, a wearable food consumption monitoring system can comprise: eyeglasses worn by a person; a camera on the eyeglasses; a spectroscopic sensor; and an electrochemical sensor, wherein the camera is triggered to record images and the spectroscopic sensor is activated to make spectroscopic scans when analysis of data from the electrochemical sensor indicates that the person is consuming food.

In another embodiment, a wearable food consumption monitoring system can comprise: eyeglasses worn by a person, wherein the eyeglasses further comprise a camera; a spectroscopic sensor; and a wrist-worn or finger-worn chewing sensor, wherein the camera is triggered to record images and the spectroscopic sensor is activated to make spectroscopic scans when analysis of data from the chewing sensor indicates that the person is consuming food. In another example, a wearable food consumption monitoring system can comprise: eyeglasses worn by a person, wherein the eyeglasses further comprise a camera; a spectroscopic sensor; and a wrist-worn or finger-worn infrared sensor, wherein the camera is triggered to record images and the spectroscopic sensor is activated to make spectroscopic scans when analysis of data from the infrared sensor indicates that the person is consuming food.

In an example, a wearable food consumption monitoring system can comprise: eyeglasses worn by a person, wherein the eyeglasses further comprise a camera; a spectroscopic sensor; and a wrist-worn or finger-worn pressure sensor, wherein the camera is triggered to record images and the spectroscopic sensor is activated to make spectroscopic scans when analysis of data from the pressure sensor indicates that the person is consuming food. In another embodiment, a wearable food consumption monitoring device can comprise: eyeglasses worn by a person; an infrared sensor on a sidepiece (e.g. a temple) of the eyeglasses, wherein the infrared sensor points toward the person's mouth; and a camera on a sidepiece (e.g. a temple) of the eyeglasses, wherein the camera points toward the person's mouth, and wherein the camera is activated to record food images when analysis of data from the infrared sensor indicates that the person is probably eating. In another example, a wearable food consumption monitoring device can comprise: eyeglasses worn by a person; an infrared sensor on a sidepiece (e.g. a temple) of the eyeglasses, wherein the infrared sensor points toward the person's mouth; at least one vibration sensor on the eyeglasses; and a camera on a sidepiece (e.g. a temple) of the eyeglasses, wherein the camera points toward the person's mouth, and wherein the camera is activated to record food images when analysis of data from the infrared sensor and the at least one vibration sensor indicates that the person is probably eating.

In an example, a wearable food consumption monitoring device can comprise: eyeglasses worn by a person; an infrared sensor on the eyeglasses, wherein the infrared sensor points toward the person's mouth; and a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor indicates that the person is probably eating. In an example, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; at least one EMG sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor and the at least one inertial motion sensor indicates that the person is probably eating. In another example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; and a camera on the eyeglasses, wherein the camera points toward the person's mouth, and wherein the camera is activated to record food images when analysis of data from the infrared sensor and the at least one inertial motion sensor indicates that the person is probably eating. In an example, a wearable food consumption monitoring device can comprise: eyeglasses worn by a person; an infrared sensor on the eyeglasses, wherein the infrared sensor points toward the person's mouth; at least one vibration sensor on the eyeglasses; and a camera on a frontpiece and/or nose bridge of the eyeglasses, wherein the camera points toward the person's mouth, and wherein the camera is activated to record food images when analysis of data from the infrared sensor and the at least one vibration sensor indicates that the person is probably eating.

In another embodiment, a wearable food consumption monitoring system can comprise: eyeglasses worn by a person, wherein the eyeglasses further comprise a camera; and a finger-worn motion sensor, wherein the camera is triggered to record food images when analysis of data from the wrist-worn motion sensor indicates that the person is consuming food. In another example, a wearable food consumption monitoring system can comprise: eyeglasses worn by a person, wherein the eyeglasses further comprise a camera; and a wrist-worn motion sensor, wherein the camera is triggered to record images along an imaging vector which points toward the person's mouth when analysis of data from the wrist-worn motion sensor indicates that the person is consuming food. In an example, a wearable food consumption monitoring device can comprise: eyeglasses worn by a person, wherein the eyeglasses further comprise a camera; and wherein the eyeglasses further comprise a chewing sensor, wherein a first camera is triggered to record images along an imaging vector which points toward the person's mouth and a second camera is triggered to record images along an imaging vector which points toward a reachable food source when analysis of data from the chewing sensor indicates that the person is consuming food.

In another embodiment, a wearable food consumption monitoring device can comprise: eyeglasses worn by a person, wherein the eyeglasses further comprise a camera; and wherein the eyeglasses further comprise a chewing sensor, wherein the camera is triggered to record images of the interaction between food and the person's mouth when analysis of data from sensor indicates that the person is consuming food. In another example, a wearable food consumption monitoring device can comprise: eyeglasses worn by a person, wherein the eyeglasses further comprise a camera; and wherein the eyeglasses further comprise a location sensor, wherein a first camera is triggered to record images along an imaging vector which points toward the person's mouth and a second camera is triggered to record images along an imaging vector which points toward a reachable food source when analysis of data from the location sensor indicates that the person is consuming food. Alternatively, a wearable food consumption monitoring device can comprise: eyeglasses worn by a person, wherein the eyeglasses further comprise a camera; and wherein the eyeglasses further comprise a motion sensor, wherein a first camera is triggered to record images along an imaging vector which points toward the person's mouth and a second camera is triggered to record images along an imaging vector which points toward a reachable food source when analysis of data from the motion sensor indicates that the person is consuming food.

In an example, a wearable food consumption monitoring device can comprise: eyeglasses worn by a person, wherein the eyeglasses further comprise a camera; and wherein the eyeglasses further comprise a optical sensor, wherein a first camera is triggered to record images along an imaging vector which points toward the person's mouth and a second camera is triggered to record images along an imaging vector which points toward a reachable food source when analysis of data from the optical sensor indicates that the person is consuming food. In another example, a wearable food consumption monitoring device can comprise: eyeglasses worn by a person, wherein the eyeglasses further comprise a camera; and wherein the eyeglasses further comprise a pressure sensor, wherein a first camera is triggered to record images along an imaging vector which points toward the person's mouth and a second camera is triggered to record images along an imaging vector which points toward a reachable food source when analysis of data from the pressure sensor indicates that the person is consuming food. In an example, a wearable food consumption monitoring device can comprise: eyeglasses worn by a person, wherein the eyeglasses further comprise a camera; and wherein the eyeglasses further comprise a proximity sensor, wherein a first camera is triggered to record images along an imaging vector which points toward the person's mouth and a second camera is triggered to record images along an imaging vector which points toward a reachable food source when analysis of data from the proximity sensor indicates that the person is consuming food.

In another embodiment, a wearable food consumption monitoring device can comprise: eyeglasses worn by a person, wherein the eyeglasses further comprise a camera; and wherein the eyeglasses further comprise a spectroscopic sensor, wherein the camera is triggered to record images of the interaction between food and the person's mouth when analysis of data from sensor indicates that the person is consuming food. Alternatively, a wearable food consumption monitoring device can comprise: eyeglasses worn by a person, wherein the eyeglasses further comprise a camera; and wherein the eyeglasses further comprise a swallow sensor, wherein a first camera is triggered to record images along an imaging vector which points toward the person's mouth and a second camera is triggered to record images along an imaging vector which points toward a reachable food source when analysis of data from the swallow sensor indicates that the person is consuming food. In another embodiment, a wearable food consumption monitoring device can comprise: eyeglasses worn by a person, wherein the eyeglasses further comprise a camera; and wherein the eyeglasses further comprise a swallowing sensor, wherein the camera is triggered to record images of the interaction between food and the person's mouth when analysis of data from sensor indicates that the person is consuming food.

In an example, a wearable food consumption monitoring device can comprise: eyeglasses worn by a person, wherein the eyeglasses further comprise a camera; and wherein the eyeglasses further comprise an electrochemical sensor, wherein a first camera is triggered to record images along an imaging vector which points toward the person's mouth and a second camera is triggered to record images along an imaging vector which points toward a reachable food source when analysis of data from the electrochemical sensor indicates that the person is consuming food. In another example, a wearable food consumption monitoring device can comprise: eyeglasses worn by a person, wherein the eyeglasses further comprise a camera; and wherein the eyeglasses further comprise an EMG sensor, wherein the camera is triggered to record images along an imaging vector which points toward the person's mouth when analysis of data from the EMG sensor indicates that the person is consuming food. In an example, a wearable food consumption monitoring device can comprise: eyeglasses worn by a person, wherein the eyeglasses further comprise a camera; and wherein the eyeglasses further comprise an infrared sensor which tracks the location of the person's hands, wherein the camera is triggered to record images along an imaging vector which points toward the person's mouth when analysis of data from the infrared sensor indicates that the person is consuming food.

In an example, a wearable food consumption monitoring device can comprise: eyeglasses worn by a person, wherein the eyeglasses further comprise a camera; and wherein the eyeglasses further comprise an infrared sensor, wherein a first camera is triggered to record images along an imaging vector which points toward the person's mouth and a second camera is triggered to record images along an imaging vector which points toward a reachable food source when analysis of data from the infrared sensor indicates that the person is consuming food. In another example, a wearable food consumption monitoring device can comprise: eyeglasses worn by a person; at least one EMG sensor on a portion of the eyeglasses which curves around the rear of the person's ear; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one EMG sensor indicates that the person is probably eating. In another embodiment, a wearable food consumption monitoring device can comprise: eyeglasses worn by a person; at least one EMG sensor on a portion of the eyeglasses which curves around the rear of the person's ear; and a camera on the eyeglasses, wherein the camera points toward the person's mouth, and wherein the camera is activated to record food images when analysis of data from the at least one EMG sensor indicates that the person is probably eating.

In an example, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on a frontpiece and/or nose bridge of the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one EMG sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one EMG sensor indicates that the person is probably eating. In another embodiment, a wearable food consumption monitoring device can comprise: eyeglasses worn by a person; at least one inertial motion sensor (e.g. gyroscope and/or accelerometer) on the eyeglasses; and a camera on a sidepiece (e.g. a temple) of the eyeglasses, wherein the camera points toward the person's mouth, and wherein the camera is activated to record food images when analysis of data from the at least one inertial motion sensor indicates that the person is probably eating.

In an example, a wearable food consumption monitoring device can comprise: eyeglasses worn by a person; at least one inertial motion sensor (e.g. gyroscope and/or accelerometer) on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one inertial motion sensor indicates that the person is probably eating. Alternatively, a wearable food consumption monitoring device can comprise: eyeglasses worn by a person; at least one vibration sensor on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one vibration sensor indicates that the person is probably eating. In an example, a wearable food consumption monitoring system can comprise: eyeglasses worn by a person; at least one wrist-worn or finger-worn inertial motion sensor (e.g. gyroscope and/or accelerometer on a smart watch or smart ring); a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one wrist-worn or finger-worn inertial motion sensor indicates that the person is probably eating.

In an example, a wearable food consumption monitoring system can comprise: eyeglasses worn by a person; at least one wrist-worn or finger-worn inertial motion sensor (e.g. gyroscope and/or accelerometer on a smart watch or smart ring); and a camera on the eyeglasses, wherein the camera points toward the person's mouth, and wherein the camera is activated to record food images when analysis of data from the at least one wrist-worn or finger-worn inertial motion sensor indicates that the person is probably eating. In another example, a wearable food consumption monitoring device can comprise: eyeglasses worn by a person, wherein the eyeglasses further comprise a camera; wherein the eyeglasses further comprise an EMG sensor; and wherein the eyeglasses further comprise an infrared sensor which tracks the location of the person's hands, wherein the camera is triggered to record images when joint analysis of data from the EMG sensor and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise a sound sensor (e.g. microphone), wherein a first camera is triggered to record images along an imaging vector which points toward the person's mouth and a second camera is triggered to record images of a reachable food source when analysis of data from the sound sensor (e.g. microphone) indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone) and an infrared sensor, wherein a first camera is triggered to record images along an imaging vector which points toward the person's mouth and a second camera is triggered to record images of a reachable food source when joint analysis of data from the sound sensor (e.g. microphone) and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a sound sensor (e.g. microphone), wherein a first camera is triggered to record images along an imaging vector which points toward the person's mouth and a second camera is triggered to record images of a reachable food source when analysis of data from the sound sensor (e.g. microphone) indicates that the person is consuming food. In another embodiment, a wearable food consumption monitoring device can comprise: eyeglasses worn by a person; wherein the eyeglasses further comprise at least two cameras; and wherein the eyeglasses further comprise a sound sensor (e.g. microphone) and an EEG sensor, wherein a first camera is triggered to record images along an imaging vector which points toward the person's mouth and a second camera is triggered to record images of a reachable food source when joint analysis of data from the sound sensor (e.g. microphone) and the EEG sensor indicates that the person is consuming food.

In an example, a wearable food consumption monitoring device can comprise: eyeglasses worn by a person; wherein the eyeglasses further comprise at least two cameras; and wherein the eyeglasses further comprise a swallow sensor and an accelerometer, wherein a first camera is triggered to record images along an imaging vector which points toward the person's mouth and a second camera is triggered to record images of a reachable food source when joint analysis of data from the swallow sensor and the accelerometer indicates that the person is consuming food. In another example, a wearable food consumption monitoring device can comprise: eyeglasses worn by a person; wherein the eyeglasses further comprise at least two cameras; and wherein the eyeglasses further comprise a swallow sensor and a motion sensor, wherein a first camera is triggered to record images along an imaging vector which points toward the person's mouth and a second camera is triggered to record images of a reachable food source when joint analysis of data from the swallow sensor and the motion sensor indicates that the person is consuming food. In an example, a wearable food consumption monitoring device can comprise: eyeglasses worn by a person; wherein the eyeglasses further comprise at least two cameras; and wherein the eyeglasses further comprise a swallow sensor, an accelerometer, and an infrared sensor, wherein a first camera is triggered to record images along an imaging vector which points toward the person's mouth and a second camera is triggered to record images of a reachable food source when joint analysis of data from the swallow sensor, the accelerometer, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor and a sound sensor (e.g. microphone), wherein a first camera is triggered to record images along an imaging vector which points toward the person's mouth and a second camera is triggered to record images of a reachable food source when joint analysis of data from the swallowing sensor and the sound sensor (e.g. microphone) indicates that the person is consuming food. In another example, a wearable food consumption monitoring device can comprise: eyeglasses worn by a person; wherein the eyeglasses further comprise at least two cameras; and wherein the eyeglasses further comprise a swallowing sensor and a motion sensor, wherein a first camera is triggered to record images along an imaging vector which points toward the person's mouth and a second camera is triggered to record images of a reachable food source when joint analysis of data from the swallowing sensor and the motion sensor indicates that the person is consuming food. In an example, a wearable food consumption monitoring device can comprise: eyeglasses worn by a person; wherein the eyeglasses further comprise at least two cameras; and wherein the eyeglasses further comprise a swallowing sensor, a sound sensor (e.g. microphone), and an infrared sensor, wherein a first camera is triggered to record images along an imaging vector which points toward the person's mouth and a second camera is triggered to record images of a reachable food source when joint analysis of data from the swallowing sensor, the sound sensor (e.g. microphone), and the infrared sensor indicates that the person is consuming food.

In another embodiment, a wearable food consumption monitoring device can comprise: eyeglasses worn by a person; wherein the eyeglasses further comprise at least two cameras; and wherein the eyeglasses further comprise an accelerometer and a chewing sensor, wherein a first camera is triggered to record images along an imaging vector which points toward the person's mouth and a second camera is triggered to record images of a reachable food source when joint analysis of data from the accelerometer and the chewing sensor indicates that the person is consuming food. In an example, a wearable food consumption monitoring device can comprise: eyeglasses worn by a person; wherein the eyeglasses further comprise at least two cameras; and wherein the eyeglasses further comprise an EEG sensor and an EMG sensor, wherein a first camera is triggered to record images along an imaging vector which points toward the person's mouth and a second camera is triggered to record images of a reachable food source when joint analysis of data from the EEG sensor and the EMG sensor indicates that the person is consuming food. In another embodiment, a wearable food consumption monitoring device can comprise: eyeglasses worn by a person; wherein the eyeglasses further comprise at least two cameras; and wherein the eyeglasses further comprise an EEG sensor, an EMG sensor, and an infrared sensor, wherein a first camera is triggered to record images along an imaging vector which points toward the person's mouth and a second camera is triggered to record images of a reachable food source when joint analysis of data from the EEG sensor, the EMG sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise an EMG sensor and a chewing sensor, wherein a first camera is triggered to record images along an imaging vector which points toward the person's mouth and a second camera is triggered to record images of a reachable food source when joint analysis of data from the EMG sensor and the chewing sensor indicates that the person is consuming food. In another example, a wearable food consumption monitoring device can comprise: eyeglasses worn by a person; wherein the eyeglasses further comprise at least two cameras; and wherein the eyeglasses further comprise an EMG sensor and an accelerometer, wherein a first camera is triggered to record images along an imaging vector which points toward the person's mouth and a second camera is triggered to record images of a reachable food source when joint analysis of data from the EMG sensor and the accelerometer indicates that the person is consuming food. In an example, a wearable food consumption monitoring device can comprise: eyeglasses worn by a person; wherein the eyeglasses further comprise at least two cameras; and wherein the eyeglasses further comprise an EMG sensor, an accelerometer, and an infrared sensor, wherein a first camera is triggered to record images along an imaging vector which points toward the person's mouth and a second camera is triggered to record images of a reachable food source when joint analysis of data from the EMG sensor, the accelerometer, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise an motion sensor and an infrared sensor, wherein a first camera is triggered to record images along an imaging vector which points toward the person's mouth and a second camera is triggered to record images of a reachable food source when joint analysis of data from the motion sensor and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone), wherein at least one camera is triggered to record images along an imaging vector which points toward the person's mouth when analysis of data from the sound sensor (e.g. microphone) indicates that the person is consuming food. In an example, a wearable food consumption monitoring device can comprise: eyeglasses worn by a person; wherein the eyeglasses further comprise one or more cameras; and wherein the eyeglasses further comprise a sound sensor (e.g. microphone) and an infrared sensor, wherein at least one camera is triggered to record images along an imaging vector which points toward the person's mouth when joint analysis of data from the sound sensor (e.g. microphone) and the infrared sensor indicates that the person is consuming food.

In another embodiment, a wearable food consumption monitoring device can comprise: eyeglasses worn by a person; wherein the eyeglasses further comprise one or more cameras; and wherein the eyeglasses further comprise a sound sensor (e.g. microphone), an accelerometer, and an infrared sensor, wherein at least one camera is triggered to record images along an imaging vector which points toward the person's mouth when joint analysis of data from the sound sensor (e.g. microphone), the accelerometer, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone) and an accelerometer, wherein at least one camera is triggered to record images along an imaging vector which points toward the person's mouth when joint analysis of data from the sound sensor (e.g. microphone) and the accelerometer indicates that the person is consuming food. Alternatively, a wearable food consumption monitoring device can comprise: eyeglasses worn by a person; wherein the eyeglasses further comprise one or more cameras; and wherein the eyeglasses further comprise a swallow sensor and an accelerometer, wherein at least one camera is triggered to record images along an imaging vector which points toward the person's mouth when joint analysis of data from the swallow sensor and the accelerometer indicates that the person is consuming food.

In another embodiment, a wearable food consumption monitoring device can comprise: eyeglasses worn by a person; wherein the eyeglasses further comprise one or more cameras; and wherein the eyeglasses further comprise a swallow sensor and a motion sensor, wherein at least one camera is triggered to record images along an imaging vector which points toward the person's mouth when joint analysis of data from the swallow sensor and the motion sensor indicates that the person is consuming food. In another example, a wearable food consumption monitoring device can comprise: eyeglasses worn by a person; wherein the eyeglasses further comprise one or more cameras; and wherein the eyeglasses further comprise a swallow sensor, an accelerometer, and an infrared sensor, wherein at least one camera is triggered to record images along an imaging vector which points toward the person's mouth when joint analysis of data from the swallow sensor, the accelerometer, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise a swallowing sensor and a sound sensor (e.g. microphone), wherein at least one camera is triggered to record images along an imaging vector which points toward the person's mouth when joint analysis of data from the swallowing sensor and the sound sensor (e.g. microphone) indicates that the person is consuming food.

In an example, a wearable food consumption monitoring device can comprise: eyeglasses worn by a person; wherein the eyeglasses further comprise one or more cameras; and wherein the eyeglasses further comprise a swallowing sensor and a motion sensor, wherein at least one camera is triggered to record images along an imaging vector which points toward the person's mouth when joint analysis of data from the swallowing sensor and the motion sensor indicates that the person is consuming food. Alternatively, a wearable food consumption monitoring device can comprise: eyeglasses worn by a person; wherein the eyeglasses further comprise one or more cameras; and wherein the eyeglasses further comprise a swallowing sensor, a chewing sensor, and an infrared sensor, wherein at least one camera is triggered to record images along an imaging vector which points toward the person's mouth when joint analysis of data from the swallowing sensor, the chewing sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise an accelerometer and a chewing sensor, wherein at least one camera is triggered to record images along an imaging vector which points toward the person's mouth when joint analysis of data from the accelerometer and the chewing sensor indicates that the person is consuming food. In another embodiment, a wearable food consumption monitoring device can comprise: eyeglasses worn by a person; wherein the eyeglasses further comprise one or more cameras; and wherein the eyeglasses further comprise an EEG sensor and an EMG sensor, wherein at least one camera is triggered to record images along an imaging vector which points toward the person's mouth when joint analysis of data from the EEG sensor and the EMG sensor indicates that the person is consuming food.

In an example, a wearable food consumption monitoring device can comprise: eyeglasses worn by a person; wherein the eyeglasses further comprise one or more cameras; and wherein the eyeglasses further comprise an EEG sensor, an EMG sensor, and an infrared sensor, wherein at least one camera is triggered to record images along an imaging vector which points toward the person's mouth when joint analysis of data from the EEG sensor, the EMG sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an EMG sensor and a chewing sensor, wherein at least one camera is triggered to record images along an imaging vector which points toward the person's mouth when joint analysis of data from the EMG sensor and the chewing sensor indicates that the person is consuming food. In another embodiment, a wearable food consumption monitoring device can comprise: eyeglasses worn by a person; wherein the eyeglasses further comprise one or more cameras; and wherein the eyeglasses further comprise an EMG sensor and an accelerometer, wherein at least one camera is triggered to record images along an imaging vector which points toward the person's mouth when joint analysis of data from the EMG sensor and the accelerometer indicates that the person is consuming food. In another example, a wearable food consumption monitoring device can comprise: eyeglasses worn by a person; wherein the eyeglasses further comprise one or more cameras; and wherein the eyeglasses further comprise an EMG sensor, an EEG sensor, and an infrared sensor, wherein at least one camera is triggered to record images along an imaging vector which points toward the person's mouth when joint analysis of data from the EMG sensor, the EEG sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise an motion sensor and an infrared sensor, wherein at least one camera is triggered to record images along an imaging vector which points toward the person's mouth when joint analysis of data from the motion sensor and the infrared sensor indicates that the person is consuming food. In another embodiment, a wearable food consumption monitoring system can comprise: eyeglasses worn by a person; a camera on the eyeglasses; a spectroscopic sensor; and a location sensor, wherein the camera is triggered to record images and the spectroscopic sensor is activated to make spectroscopic scans when analysis of data from the location sensor indicates that the person is consuming food. In another example, a wearable food consumption monitoring system can comprise: eyeglasses worn by a person; a camera on the eyeglasses; a spectroscopic sensor; and a smell sensor, wherein the camera is triggered to record images and the spectroscopic sensor is activated to make spectroscopic scans when analysis of data from the smell sensor indicates that the person is consuming food. In an example, a wearable food consumption monitoring system can comprise: eyeglasses worn by a person; a camera on the eyeglasses; a spectroscopic sensor; and an EMG sensor, wherein the camera is triggered to record images and the spectroscopic sensor is activated to make spectroscopic scans when analysis of data from the EMG sensor indicates that the person is consuming food.

In an example, a wearable food consumption monitoring system can comprise: eyeglasses worn by a person, wherein the eyeglasses further comprise a camera; a spectroscopic sensor; and a wrist-worn or finger-worn location sensor, wherein the camera is triggered to record images and the spectroscopic sensor is activated to make spectroscopic scans when analysis of data from the location sensor indicates that the person is consuming food. In another example, a wearable food consumption monitoring system can comprise: eyeglasses worn by a person, wherein the eyeglasses further comprise a camera; a spectroscopic sensor; and a wrist-worn or finger-worn proximity sensor, wherein the camera is triggered to record images and the spectroscopic sensor is activated to make spectroscopic scans when analysis of data from the proximity sensor indicates that the person is consuming food. In an example, a wearable food consumption monitoring device can comprise: eyeglasses worn by a person; an infrared sensor on a sidepiece (e.g. a temple) of the eyeglasses, wherein the infrared sensor points toward the person's mouth; at least one EMG sensor on a portion of the eyeglasses which curves around the rear of the person's ear; and a camera on a sidepiece (e.g. a temple) of the eyeglasses, wherein the camera points toward the person's mouth, and wherein the camera is activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the infrared sensor indicates that the person is probably eating.

In another embodiment, a wearable food consumption monitoring device can comprise: eyeglasses worn by a person; an infrared sensor on the eyeglasses, wherein the infrared sensor points toward the person's mouth; and a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor indicates that the person is probably eating. In another example, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; and a camera on a frontpiece and/or nose bridge of the eyeglasses, wherein the camera points toward the person's mouth, and wherein the camera is activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor and the at least one EMG sensor indicates that the person is probably eating. In another embodiment, a wearable food consumption monitoring device can comprise: eyeglasses worn by a person; an infrared sensor on the eyeglasses, wherein the infrared sensor points toward the person's mouth; at least one EMG sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the infrared sensor and the at least one inertial motion sensor indicates that the person is probably eating. In another example, a wearable food consumption monitoring device can comprise: eyeglasses worn by a person; an infrared sensor on the eyeglasses, wherein the infrared sensor points toward the person's mouth; at least one vibration sensor on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor and the at least one vibration sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one vibration sensor on the eyeglasses; and a camera on the eyeglasses, wherein the camera points toward the person's mouth, and wherein the camera is activated to record food images when analysis of data from the infrared sensor and the at least one vibration sensor indicates that the person is probably eating. In an example, a wearable food consumption monitoring system can comprise: eyeglasses worn by a person, wherein the eyeglasses further comprise a camera; and a finger-worn motion sensor, wherein the camera is triggered to record images along an imaging vector which points toward the person's mouth when analysis of data from the wrist-worn motion sensor indicates that the person is consuming food.

In an example, a wearable food consumption monitoring system can comprise: eyeglasses worn by a person, wherein the eyeglasses further comprise a camera; and a wrist-worn motion sensor, wherein the camera is triggered to record food images when analysis of data from the wrist-worn motion sensor indicates that the person is consuming food. In another example, a wearable food consumption monitoring device can comprise: eyeglasses worn by a person, wherein the eyeglasses further comprise a camera; and wherein the eyeglasses further comprise a chewing sensor, wherein the camera is triggered to record images along an imaging vector which points toward the person's mouth when analysis of data from the chewing sensor indicates that the person is consuming food. In an example, a wearable food consumption monitoring device can comprise: eyeglasses worn by a person, wherein the eyeglasses further comprise a camera; and wherein the eyeglasses further comprise a GPS sensor, wherein a first camera is triggered to record images along an imaging vector which points toward the person's mouth and a second camera is triggered to record images along an imaging vector which points toward a reachable food source when analysis of data from the GPS sensor indicates that the person is consuming food.

In an example, a wearable food consumption monitoring device can comprise: eyeglasses worn by a person, wherein the eyeglasses further comprise a camera; and wherein the eyeglasses further comprise a location sensor, wherein the camera is triggered to record images along an imaging vector which points toward the person's mouth when analysis of data from the location sensor indicates that the person is consuming food. In another example, a wearable food consumption monitoring device can comprise: eyeglasses worn by a person, wherein the eyeglasses further comprise a camera; and wherein the eyeglasses further comprise a motion sensor, wherein the camera is triggered to record images along an imaging vector which points toward the person's mouth when analysis of data from the motion sensor indicates that the person is consuming food. In another embodiment, a wearable food consumption monitoring device can comprise: eyeglasses worn by a person, wherein the eyeglasses further comprise a camera; and wherein the eyeglasses further comprise a piezoelectric sensor, wherein a first camera is triggered to record images along an imaging vector which points toward the person's mouth and a second camera is triggered to record images along an imaging vector which points toward a reachable food source when analysis of data from the piezoelectric sensor indicates that the person is consuming food.

In an example, a wearable food consumption monitoring device can comprise: eyeglasses worn by a person, wherein the eyeglasses further comprise a camera; and wherein the eyeglasses further comprise a pressure sensor, wherein the camera is triggered to record images along an imaging vector which points toward the person's mouth when analysis of data from the pressure sensor indicates that the person is consuming food. In another example, a wearable food consumption monitoring device can comprise: eyeglasses worn by a person, wherein the eyeglasses further comprise a camera; and wherein the eyeglasses further comprise a smell sensor, wherein a first camera is triggered to record images along an imaging vector which points toward the person's mouth and a second camera is triggered to record images along an imaging vector which points toward a reachable food source when analysis of data from the smell sensor indicates that the person is consuming food. In another embodiment, a wearable food consumption monitoring device can comprise: eyeglasses worn by a person, wherein the eyeglasses further comprise a camera; and wherein the eyeglasses further comprise a strain gauge, wherein a first camera is triggered to record images along an imaging vector which points toward the person's mouth and a second camera is triggered to record images along an imaging vector which points toward a reachable food source when analysis of data from the strain gauge indicates that the person is consuming food.

In an example, a wearable food consumption monitoring device can comprise: eyeglasses worn by a person, wherein the eyeglasses further comprise a camera; and wherein the eyeglasses further comprise a swallowing sensor, wherein the camera is triggered to record images along an imaging vector which points toward the person's mouth when analysis of data from the swallowing sensor indicates that the person is consuming food. In another embodiment, a wearable food consumption monitoring device can comprise: eyeglasses worn by a person, wherein the eyeglasses further comprise a camera; and wherein the eyeglasses further comprise an EEG sensor, wherein a first camera is triggered to record images along an imaging vector which points toward the person's mouth and a second camera is triggered to record images along an imaging vector which points toward a reachable food source when analysis of data from the EEG sensor indicates that the person is consuming food. In an example, a wearable food consumption monitoring device can comprise: eyeglasses worn by a person, wherein the eyeglasses further comprise a camera; and wherein the eyeglasses further comprise an electrochemical sensor, wherein the camera is triggered to record images along an imaging vector which points toward the person's mouth when analysis of data from the electrochemical sensor indicates that the person is consuming food.

In an example, a wearable food consumption monitoring device can comprise: eyeglasses worn by a person, wherein the eyeglasses further comprise a camera; and wherein the eyeglasses further comprise an EMG sensor, wherein the camera is triggered to record images of the interaction between food and the person's mouth when analysis of data from sensor indicates that the person is consuming food. In another example, a wearable food consumption monitoring device can comprise: eyeglasses worn by a person, wherein the eyeglasses further comprise a camera; and wherein the eyeglasses further comprise an infrared sensor which tracks the location of the person's hands, wherein the camera is triggered to record images when analysis of data from the infrared sensor indicates that the person is consuming food. Alternatively, a wearable food consumption monitoring device can comprise: eyeglasses worn by a person, wherein the eyeglasses further comprise a camera; and wherein the eyeglasses further comprise an optical sensor, wherein the camera is triggered to record images along an imaging vector which points toward the person's mouth when analysis of data from the optical sensor indicates that the person is consuming food.

In an example, a wearable food consumption monitoring device can comprise: eyeglasses worn by a person; at least one EMG sensor on a portion of the eyeglasses which curves around the rear of the person's ear; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one EMG sensor indicates that the person is probably eating. In another example, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one EMG sensor indicates that the person is probably eating. In another embodiment, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on a sidepiece (e.g. a temple) of the eyeglasses, wherein the camera points toward the person's mouth, and wherein the camera is activated to record food images when analysis of data from the at least one EMG sensor indicates that the person is probably eating.

In an example, a wearable food consumption monitoring device can comprise: eyeglasses worn by a person; at least one EMG sensor on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one EMG sensor indicates that the person is probably eating. In another example, a wearable food consumption monitoring device can comprise: eyeglasses worn by a person; at least one inertial motion sensor (e.g. gyroscope and/or accelerometer) on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one inertial motion sensor indicates that the person is probably eating. In another embodiment, a wearable food consumption monitoring device can comprise: eyeglasses worn by a person; at least one inertial motion sensor (e.g. gyroscope and/or accelerometer) on the eyeglasses; and a camera on the eyeglasses, wherein the camera points toward the person's mouth, and wherein the camera is activated to record food images when analysis of data from the at least one inertial motion sensor indicates that the person is probably eating.

In an example, a wearable food consumption monitoring device can comprise: eyeglasses worn by a person; at least one vibration sensor on the eyeglasses; and a camera on a frontpiece and/or nose bridge of the eyeglasses, wherein the camera points toward the person's mouth, and wherein the camera is activated to record food images when analysis of data from the at least one vibration sensor indicates that the person is probably eating. In another example, a wearable food consumption monitoring system can comprise: eyeglasses worn by a person; at least one wrist-worn or finger-worn inertial motion sensor (e.g. gyroscope and/or accelerometer on a smart watch or smart ring); a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one wrist-worn or finger-worn inertial motion sensor indicates that the person is probably eating. In another embodiment, a wearable food consumption monitoring device can comprise: eyeglasses worn by a person, wherein the eyeglasses further comprise a camera; wherein the eyeglasses further comprise a motion sensor; and wherein the eyeglasses further comprise an infrared sensor which tracks the location of the person's hands, wherein the camera is triggered to record images along an imaging vector which points toward the person's mouth when joint analysis of data from the motion sensor and the infrared sensor indicates that the person is consuming food.

In an example, a wearable food consumption monitoring device can comprise: eyeglasses worn by a person, wherein the eyeglasses further comprise a camera; wherein the eyeglasses further comprise an EMG sensor; and wherein the eyeglasses further comprise an infrared sensor which tracks the location of the person's hands, wherein the camera is triggered to record images along an imaging vector which points toward the person's mouth when joint analysis of data from the EMG sensor and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise a sound sensor (e.g. microphone) and an accelerometer, wherein a first camera is triggered to record images along an imaging vector which points toward the person's mouth and a second camera is triggered to record images of a reachable food source when joint analysis of data from the sound sensor (e.g. microphone) and the accelerometer indicates that the person is consuming food. Alternatively, a wearable food consumption monitoring device can comprise: eyeglasses worn by a person; wherein the eyeglasses further comprise at least two cameras; and wherein the eyeglasses further comprise a sound sensor (e.g. microphone), an accelerometer, and an infrared sensor, wherein a first camera is triggered to record images along an imaging vector which points toward the person's mouth and a second camera is triggered to record images of a reachable food source when joint analysis of data from the sound sensor (e.g. microphone), the accelerometer, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone), an accelerometer, and an infrared sensor, wherein a first camera is triggered to record images along an imaging vector which points toward the person's mouth and a second camera is triggered to record images of a reachable food source when joint analysis of data from the sound sensor (e.g. microphone), the accelerometer, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a sound sensor (e.g. microphone) and a chewing sensor, wherein a first camera is triggered to record images along an imaging vector which points toward the person's mouth and a second camera is triggered to record images of a reachable food source when joint analysis of data from the sound sensor (e.g. microphone) and the chewing sensor indicates that the person is consuming food. In another embodiment, a wearable food consumption monitoring device can comprise: eyeglasses worn by a person; wherein the eyeglasses further comprise at least two cameras; and wherein the eyeglasses further comprise a swallow sensor and a chewing sensor, wherein a first camera is triggered to record images along an imaging vector which points toward the person's mouth and a second camera is triggered to record images of a reachable food source when joint analysis of data from the swallow sensor and the chewing sensor indicates that the person is consuming food.

In an example, a wearable food consumption monitoring device can comprise: eyeglasses worn by a person; wherein the eyeglasses further comprise at least two cameras; and wherein the eyeglasses further comprise a swallow sensor and an infrared sensor, wherein a first camera is triggered to record images along an imaging vector which points toward the person's mouth and a second camera is triggered to record images of a reachable food source when joint analysis of data from the swallow sensor and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a swallow sensor, an EEG sensor, and an infrared sensor, wherein a first camera is triggered to record images along an imaging vector which points toward the person's mouth and a second camera is triggered to record images of a reachable food source when joint analysis of data from the swallow sensor, the EEG sensor, and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise a swallowing sensor and an EEG sensor, wherein a first camera is triggered to record images along an imaging vector which points toward the person's mouth and a second camera is triggered to record images of a reachable food source when joint analysis of data from the swallowing sensor and the EEG sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor, wherein a first camera is triggered to record images along an imaging vector which points toward the person's mouth and a second camera is triggered to record images of a reachable food source when analysis of data from the swallowing sensor indicates that the person is consuming food. In another example, a wearable food consumption monitoring device can comprise: eyeglasses worn by a person; wherein the eyeglasses further comprise at least two cameras; and wherein the eyeglasses further comprise a swallowing sensor, a motion sensor, and an infrared sensor, wherein a first camera is triggered to record images along an imaging vector which points toward the person's mouth and a second camera is triggered to record images of a reachable food source when joint analysis of data from the swallowing sensor, the motion sensor, and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise an accelerometer, a chewing sensor, and an infrared sensor, wherein a first camera is triggered to record images along an imaging vector which points toward the person's mouth and a second camera is triggered to record images of a reachable food source when joint analysis of data from the accelerometer, the chewing sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise an EEG sensor and a motion sensor, wherein a first camera is triggered to record images along an imaging vector which points toward the person's mouth and a second camera is triggered to record images of a reachable food source when joint analysis of data from the EEG sensor and the motion sensor indicates that the person is consuming food. In another example, a wearable food consumption monitoring device can comprise: eyeglasses worn by a person; wherein the eyeglasses further comprise at least two cameras; and wherein the eyeglasses further comprise an EEG sensor, a motion sensor, and an infrared sensor, wherein a first camera is triggered to record images along an imaging vector which points toward the person's mouth and a second camera is triggered to record images of a reachable food source when joint analysis of data from the EEG sensor, the motion sensor, and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise an EMG sensor and an EEG sensor, wherein a first camera is triggered to record images along an imaging vector which points toward the person's mouth and a second camera is triggered to record images of a reachable food source when joint analysis of data from the EMG sensor and the EEG sensor indicates that the person is consuming food.

In an example, a wearable food consumption monitoring device can comprise: eyeglasses worn by a person; wherein the eyeglasses further comprise at least two cameras; and wherein the eyeglasses further comprise an EMG sensor, a chewing sensor, and an infrared sensor, wherein a first camera is triggered to record images along an imaging vector which points toward the person's mouth and a second camera is triggered to record images of a reachable food source when joint analysis of data from the EMG sensor, the chewing sensor, and the infrared sensor indicates that the person is consuming food. Alternatively, a wearable food consumption monitoring device can comprise: eyeglasses worn by a person; wherein the eyeglasses further comprise at least two cameras; and wherein the eyeglasses further comprise an EMG sensor, wherein a first camera is triggered to record images along an imaging vector which points toward the person's mouth and a second camera is triggered to record images of a reachable food source when analysis of data from the EMG sensor indicates that the person is consuming food. In another example, a wearable food consumption monitoring device can comprise: eyeglasses worn by a person; wherein the eyeglasses further comprise at least two cameras; and wherein the eyeglasses further comprise an motion sensor, a chewing sensor, and an infrared sensor, wherein a first camera is triggered to record images along an imaging vector which points toward the person's mouth and a second camera is triggered to record images of a reachable food source when joint analysis of data from the motion sensor, the chewing sensor, and the infrared sensor indicates that the person is consuming food.

In another embodiment, a wearable food consumption monitoring device can comprise: eyeglasses worn by a person; wherein the eyeglasses further comprise one or more cameras; and wherein the eyeglasses further comprise a sound sensor (e.g. microphone) and an accelerometer, wherein at least one camera is triggered to record images along an imaging vector which points toward the person's mouth when joint analysis of data from the sound sensor (e.g. microphone) and the accelerometer indicates that the person is consuming food. In an example, a wearable food consumption monitoring device can comprise: eyeglasses worn by a person; wherein the eyeglasses further comprise one or more cameras; and wherein the eyeglasses further comprise a sound sensor (e.g. microphone), an accelerometer, and an infrared sensor, wherein at least one camera is triggered to record images along an imaging vector which points toward the person's mouth when joint analysis of data from the sound sensor (e.g. microphone), the accelerometer, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone), an EEG sensor, and an infrared sensor, wherein at least one camera is triggered to record images along an imaging vector which points toward the person's mouth when joint analysis of data from the sound sensor (e.g. microphone), the EEG sensor, and the infrared sensor indicates that the person is consuming food.

In another embodiment, a wearable food consumption monitoring device can comprise: eyeglasses worn by a person; wherein the eyeglasses further comprise one or more cameras; and wherein the eyeglasses further comprise a sound sensor (e.g. microphone), wherein at least one camera is triggered to record images along an imaging vector which points toward the person's mouth when analysis of data from the sound sensor (e.g. microphone) indicates that the person is consuming food. In an example, a wearable food consumption monitoring device can comprise: eyeglasses worn by a person; wherein the eyeglasses further comprise one or more cameras; and wherein the eyeglasses further comprise a swallow sensor and a chewing sensor, wherein at least one camera is triggered to record images along an imaging vector which points toward the person's mouth when joint analysis of data from the swallow sensor and the chewing sensor indicates that the person is consuming food. In another example, a wearable food consumption monitoring device can comprise: eyeglasses worn by a person; wherein the eyeglasses further comprise one or more cameras; and wherein the eyeglasses further comprise a swallow sensor and an infrared sensor, wherein at least one camera is triggered to record images along an imaging vector which points toward the person's mouth when joint analysis of data from the swallow sensor and the infrared sensor indicates that the person is consuming food.

In another embodiment, a wearable food consumption monitoring device can comprise: eyeglasses worn by a person; wherein the eyeglasses further comprise one or more cameras; and wherein the eyeglasses further comprise a swallow sensor, an EEG sensor, and an infrared sensor, wherein at least one camera is triggered to record images along an imaging vector which points toward the person's mouth when joint analysis of data from the swallow sensor, the EEG sensor, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise a swallowing sensor and an EEG sensor, wherein at least one camera is triggered to record images along an imaging vector which points toward the person's mouth when joint analysis of data from the swallowing sensor and the EEG sensor indicates that the person is consuming food. In another example, a wearable food consumption monitoring device can comprise: eyeglasses worn by a person; wherein the eyeglasses further comprise one or more cameras; and wherein the eyeglasses further comprise a swallowing sensor, an EEG sensor, and an infrared sensor, wherein at least one camera is triggered to record images along an imaging vector which points toward the person's mouth when joint analysis of data from the swallowing sensor, the EEG sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallowing sensor, wherein at least one camera is triggered to record images along an imaging vector which points toward the person's mouth when analysis of data from the swallowing sensor indicates that the person is consuming food. In an example, a wearable food consumption monitoring device can comprise: eyeglasses worn by a person; wherein the eyeglasses further comprise one or more cameras; and wherein the eyeglasses further comprise an accelerometer, a chewing sensor, and an infrared sensor, wherein at least one camera is triggered to record images along an imaging vector which points toward the person's mouth when joint analysis of data from the accelerometer, the chewing sensor, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise an EEG sensor and a motion sensor, wherein at least one camera is triggered to record images along an imaging vector which points toward the person's mouth when joint analysis of data from the EEG sensor and the motion sensor indicates that the person is consuming food.

In another embodiment, a wearable food consumption monitoring device can comprise: eyeglasses worn by a person; wherein the eyeglasses further comprise one or more cameras; and wherein the eyeglasses further comprise an EEG sensor, a motion sensor, and an infrared sensor, wherein at least one camera is triggered to record images along an imaging vector which points toward the person's mouth when joint analysis of data from the EEG sensor, the motion sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an EMG sensor and an EEG sensor, wherein at least one camera is triggered to record images along an imaging vector which points toward the person's mouth when joint analysis of data from the EMG sensor and the EEG sensor indicates that the person is consuming food. Alternatively, a wearable food consumption monitoring device can comprise: eyeglasses worn by a person; wherein the eyeglasses further comprise one or more cameras; and wherein the eyeglasses further comprise an EMG sensor and an infrared sensor, wherein at least one camera is triggered to record images along an imaging vector which points toward the person's mouth when joint analysis of data from the EMG sensor and the infrared sensor indicates that the person is consuming food.

In another embodiment, a wearable food consumption monitoring device can comprise: eyeglasses worn by a person; wherein the eyeglasses further comprise one or more cameras; and wherein the eyeglasses further comprise an EMG sensor, an accelerometer, and an infrared sensor, wherein at least one camera is triggered to record images along an imaging vector which points toward the person's mouth when joint analysis of data from the EMG sensor, the accelerometer, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an motion sensor and a chewing sensor, wherein at least one camera is triggered to record images along an imaging vector which points toward the person's mouth when joint analysis of data from the motion sensor and the chewing sensor indicates that the person is consuming food. In an example, a wearable food consumption monitoring system can comprise: eyeglasses worn by a person; a camera on the eyeglasses; a spectroscopic sensor; and a motion sensor, wherein the camera is triggered to record images and the spectroscopic sensor is activated to make spectroscopic scans when analysis of data from the motion sensor indicates that the person is consuming food.

In another embodiment, a wearable food consumption monitoring system can comprise: eyeglasses worn by a person; a camera on the eyeglasses; a spectroscopic sensor; and a strain gauge, wherein the camera is triggered to record images and the spectroscopic sensor is activated to make spectroscopic scans when analysis of data from the strain gauge indicates that the person is consuming food. In another example, a wearable food consumption monitoring system can comprise: eyeglasses worn by a person; a camera on the eyeglasses; a spectroscopic sensor; and an infrared sensor, wherein the camera is triggered to record images and the spectroscopic sensor is activated to make spectroscopic scans when analysis of data from the infrared sensor indicates that the person is consuming food.

In an example, a wearable food consumption monitoring system can comprise: eyeglasses worn by a person, wherein the eyeglasses further comprise a camera; a spectroscopic sensor; and a wrist-worn or finger-worn electrochemical sensor, wherein the camera is triggered to record images and the spectroscopic sensor is activated to make spectroscopic scans when analysis of data from the electrochemical sensor indicates that the person is consuming food. Alternatively, a wearable food consumption monitoring system can comprise: eyeglasses worn by a person, wherein the eyeglasses further comprise a camera; a spectroscopic sensor; and a wrist-worn or finger-worn motion sensor, wherein the camera is triggered to record images and the spectroscopic sensor is activated to make spectroscopic scans when analysis of data from the motion sensor indicates that the person is consuming food.

In an example, a wearable food consumption monitoring system can comprise: eyeglasses worn by a person, wherein the eyeglasses further comprise a camera; a spectroscopic sensor; and a wrist-worn or finger-worn smell sensor, wherein the camera is triggered to record images and the spectroscopic sensor is activated to make spectroscopic scans when analysis of data from the smell sensor indicates that the person is consuming food. In another example, a wearable food consumption monitoring device can comprise: eyeglasses worn by a person; an infrared sensor on a sidepiece (e.g. a temple) of the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on a sidepiece (e.g. a temple) of the eyeglasses, wherein the camera points toward the person's mouth, and wherein the camera is activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor indicates that the person is probably eating. In another embodiment, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; and a camera on the eyeglasses, wherein the camera points toward the person's mouth, and wherein the camera is activated to record food images when analysis of data from the infrared sensor and the at least one EMG sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on a frontpiece and/or nose bridge of the eyeglasses, wherein the camera points toward the person's mouth, and wherein the camera is activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; at least one EMG sensor on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor and the at least one EMG sensor indicates that the person is probably eating. In another example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor and the at least one inertial motion sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one vibration sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the infrared sensor and the at least one vibration sensor indicates that the person is probably eating. In an example, a wearable food consumption monitoring system can comprise: eyeglasses worn by a person, wherein the eyeglasses further comprise at least two cameras; and a finger-worn motion sensor (e.g. in a smart ring), wherein a first camera is triggered to record images along an imaging vector which points toward the person's mouth and a second camera is triggered to record images of a reachable food source when analysis of data from the finger-worn motion sensor indicates that the person is consuming food. In another example, a wearable food consumption monitoring device can comprise: eyeglasses worn by a person, wherein the eyeglasses further comprise a camera; and wherein the eyeglasses further comprise a blood pressure sensor, wherein a first camera is triggered to record images along an imaging vector which points toward the person's mouth and a second camera is triggered to record images along an imaging vector which points toward a reachable food source when analysis of data from the blood pressure sensor indicates that the person is consuming food.

In an example, a wearable food consumption monitoring system can comprise: eyeglasses worn by a person, wherein the eyeglasses further comprise a camera; and a finger-worn motion sensor, wherein the camera is triggered to record food images when analysis of data from the wrist-worn motion sensor indicates that the person is consuming food. In an example, a wearable food consumption monitoring device can comprise: eyeglasses worn by a person, wherein the eyeglasses further comprise a camera; and wherein the eyeglasses further comprise a chewing sensor, wherein the camera is triggered to record images of the interaction between food and the person's mouth when analysis of data from sensor indicates that the person is consuming food. In another example, a wearable food consumption monitoring device can comprise: eyeglasses worn by a person, wherein the eyeglasses further comprise a camera; and wherein the eyeglasses further comprise a GPS sensor, wherein the camera is triggered to record images along an imaging vector which points toward the person's mouth when analysis of data from the GPS sensor indicates that the person is consuming food.

In an example, a wearable food consumption monitoring device can comprise: eyeglasses worn by a person, wherein the eyeglasses further comprise a camera; and wherein the eyeglasses further comprise a location sensor, wherein a first camera is triggered to record images along an imaging vector which points toward the person's mouth and a second camera is triggered to record images along an imaging vector which points toward a reachable food source when analysis of data from the location sensor indicates that the person is consuming food. In another embodiment, a wearable food consumption monitoring device can comprise: eyeglasses worn by a person, wherein the eyeglasses further comprise a camera; and wherein the eyeglasses further comprise a motion sensor, wherein the camera is triggered to record images of the interaction between food and the person's mouth when analysis of data from sensor indicates that the person is consuming food.

In an example, a wearable food consumption monitoring device can comprise: eyeglasses worn by a person, wherein the eyeglasses further comprise a camera; and wherein the eyeglasses further comprise a piezoelectric sensor, wherein the camera is triggered to record images along an imaging vector which points toward the person's mouth when analysis of data from the piezoelectric sensor indicates that the person is consuming food. In another example, a wearable food consumption monitoring device can comprise: eyeglasses worn by a person, wherein the eyeglasses further comprise a camera; and wherein the eyeglasses further comprise a pressure sensor, wherein a first camera is triggered to record images along an imaging vector which points toward the person's mouth and a second camera is triggered to record images along an imaging vector which points toward a reachable food source when analysis of data from the pressure sensor indicates that the person is consuming food. In another embodiment, a wearable food consumption monitoring device can comprise: eyeglasses worn by a person, wherein the eyeglasses further comprise a camera; and wherein the eyeglasses further comprise a smell sensor, wherein the camera is triggered to record images along an imaging vector which points toward the person's mouth when analysis of data from the smell sensor indicates that the person is consuming food.

In an example, a wearable food consumption monitoring device can comprise: eyeglasses worn by a person, wherein the eyeglasses further comprise a camera; and wherein the eyeglasses further comprise a strain gauge, wherein the camera is triggered to record images along an imaging vector which points toward the person's mouth when analysis of data from the strain gauge indicates that the person is consuming food. In another embodiment, a wearable food consumption monitoring device can comprise: eyeglasses worn by a person, wherein the eyeglasses further comprise a camera; and wherein the eyeglasses further comprise a swallowing sensor, wherein the camera is triggered to record images of the interaction between food and the person's mouth when analysis of data from sensor indicates that the person is consuming food. In another example, a wearable food consumption monitoring device can comprise: eyeglasses worn by a person, wherein the eyeglasses further comprise a camera; and wherein the eyeglasses further comprise an EEG sensor, wherein the camera is triggered to record images along an imaging vector which points toward the person's mouth when analysis of data from the EEG sensor indicates that the person is consuming food.

In an example, a wearable food consumption monitoring device can comprise: eyeglasses worn by a person, wherein the eyeglasses further comprise a camera; and wherein the eyeglasses further comprise an electrochemical sensor, wherein a first camera is triggered to record images along an imaging vector which points toward the person's mouth and a second camera is triggered to record images along an imaging vector which points toward a reachable food source when analysis of data from the electrochemical sensor indicates that the person is consuming food. Alternatively, a wearable food consumption monitoring device can comprise: eyeglasses worn by a person, wherein the eyeglasses further comprise a camera; and wherein the eyeglasses further comprise an EMG sensor, wherein a first camera is triggered to record images along an imaging vector which points toward the person's mouth and a second camera is triggered to record images along an imaging vector which points toward a reachable food source when analysis of data from the EMG sensor indicates that the person is consuming food. In another example, a wearable food consumption monitoring device can comprise: eyeglasses worn by a person, wherein the eyeglasses further comprise a camera; and wherein the eyeglasses further comprise an infrared sensor which tracks the location of the person's hands, wherein the camera is triggered to record images along an imaging vector which points toward the person's mouth when analysis of data from the infrared sensor indicates that the person is consuming food.

In an example, a wearable food consumption monitoring system can comprise: eyeglasses worn by a person, wherein the eyeglasses further comprise at least two cameras; and wrist-worn motion sensor (e.g. in a smart watch), wherein a first camera is triggered to record images along an imaging vector which points toward the person's mouth and a second camera is triggered to record images of a reachable food source when analysis of data from the wrist-worn motion sensor indicates that the person is consuming food. In another example, a wearable food consumption monitoring device can comprise: eyeglasses worn by a person; at least one EMG sensor on a portion of the eyeglasses which curves around the rear of the person's ear; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one EMG sensor indicates that the person is probably eating. In an example, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one EMG sensor indicates that the person is probably eating.

In another embodiment, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one EMG sensor on the eyeglasses; and a camera on a frontpiece and/or nose bridge of the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one inertial motion sensor (e.g. gyroscope and/or accelerometer) on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one inertial motion sensor indicates that the person is probably eating.

In an example, a wearable food consumption monitoring device can comprise: eyeglasses worn by a person; at least one vibration sensor on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one vibration sensor indicates that the person is probably eating. In another example, a wearable food consumption monitoring device can comprise: eyeglasses worn by a person; at least one vibration sensor on the eyeglasses; and a camera on a sidepiece (e.g. a temple) of the eyeglasses, wherein the camera points toward the person's mouth, and wherein the camera is activated to record food images when analysis of data from the at least one vibration sensor indicates that the person is probably eating. In another embodiment, a wearable food consumption monitoring system can comprise: eyeglasses worn by a person; at least one wrist-worn or finger-worn inertial motion sensor (e.g. gyroscope and/or accelerometer on a smart watch or smart ring); a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one wrist-worn or finger-worn inertial motion sensor indicates that the person is probably eating.

In an example, a wearable food consumption monitoring device can comprise: eyeglasses worn by a person, wherein the eyeglasses further comprise a camera; wherein the eyeglasses further comprise a motion sensor; and wherein the eyeglasses further comprise an infrared sensor which tracks the location of the person's hands, wherein the camera is triggered to record images when joint analysis of data from the motion sensor and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a chewing sensor and an infrared sensor, wherein a first camera is triggered to record images along an imaging vector which points toward the person's mouth and a second camera is triggered to record images of a reachable food source when joint analysis of data from the chewing sensor and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise a sound sensor (e.g. microphone) and a chewing sensor, wherein a first camera is triggered to record images along an imaging vector which points toward the person's mouth and a second camera is triggered to record images of a reachable food source when joint analysis of data from the sound sensor (e.g. microphone) and the chewing sensor indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone), a chewing sensor, and an infrared sensor, wherein a first camera is triggered to record images along an imaging vector which points toward the person's mouth and a second camera is triggered to record images of a reachable food source when joint analysis of data from the sound sensor (e.g. microphone), the chewing sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a sound sensor (e.g. microphone), an EEG sensor, and an infrared sensor, wherein a first camera is triggered to record images along an imaging vector which points toward the person's mouth and a second camera is triggered to record images of a reachable food source when joint analysis of data from the sound sensor (e.g. microphone), the EEG sensor, and the infrared sensor indicates that the person is consuming food. Alternatively, a wearable food consumption monitoring device can comprise: eyeglasses worn by a person; wherein the eyeglasses further comprise at least two cameras; and wherein the eyeglasses further comprise a sound sensor (e.g. microphone) and an accelerometer, wherein a first camera is triggered to record images along an imaging vector which points toward the person's mouth and a second camera is triggered to record images of a reachable food source when joint analysis of data from the sound sensor (e.g. microphone) and the accelerometer indicates that the person is consuming food.

In an example, a wearable food consumption monitoring device can comprise: eyeglasses worn by a person; wherein the eyeglasses further comprise at least two cameras; and wherein the eyeglasses further comprise a swallow sensor and an EEG sensor, wherein a first camera is triggered to record images along an imaging vector which points toward the person's mouth and a second camera is triggered to record images of a reachable food source when joint analysis of data from the swallow sensor and the EEG sensor indicates that the person is consuming food. In another example, a wearable food consumption monitoring device can comprise: eyeglasses worn by a person; wherein the eyeglasses further comprise at least two cameras; and wherein the eyeglasses further comprise a swallow sensor, a chewing sensor, and an infrared sensor, wherein a first camera is triggered to record images along an imaging vector which points toward the person's mouth and a second camera is triggered to record images of a reachable food source when joint analysis of data from the swallow sensor, the chewing sensor, and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise a swallow sensor, an EMG sensor, and an infrared sensor, wherein a first camera is triggered to record images along an imaging vector which points toward the person's mouth and a second camera is triggered to record images of a reachable food source when joint analysis of data from the swallow sensor, the EMG sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor and an EMG sensor, wherein a first camera is triggered to record images along an imaging vector which points toward the person's mouth and a second camera is triggered to record images of a reachable food source when joint analysis of data from the swallowing sensor and the EMG sensor indicates that the person is consuming food. In another embodiment, a wearable food consumption monitoring device can comprise: eyeglasses worn by a person; wherein the eyeglasses further comprise at least two cameras; and wherein the eyeglasses further comprise a swallowing sensor, an EEG sensor, and an infrared sensor, wherein a first camera is triggered to record images along an imaging vector which points toward the person's mouth and a second camera is triggered to record images of a reachable food source when joint analysis of data from the swallowing sensor, the EEG sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor, a chewing sensor, and an infrared sensor, wherein a first camera is triggered to record images along an imaging vector which points toward the person's mouth and a second camera is triggered to record images of a reachable food source when joint analysis of data from the swallowing sensor, the chewing sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise an accelerometer, wherein a first camera is triggered to record images along an imaging vector which points toward the person's mouth and a second camera is triggered to record images of a reachable food source when analysis of data from the accelerometer indicates that the person is consuming food.

In an example, a wearable food consumption monitoring device can comprise: eyeglasses worn by a person; wherein the eyeglasses further comprise at least two cameras; and wherein the eyeglasses further comprise an EEG sensor and an infrared sensor, wherein a first camera is triggered to record images along an imaging vector which points toward the person's mouth and a second camera is triggered to record images of a reachable food source when joint analysis of data from the EEG sensor and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise an EEG sensor, an accelerometer, and an infrared sensor, wherein a first camera is triggered to record images along an imaging vector which points toward the person's mouth and a second camera is triggered to record images of a reachable food source when joint analysis of data from the EEG sensor, the accelerometer, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise an EMG sensor and a motion sensor, wherein a first camera is triggered to record images along an imaging vector which points toward the person's mouth and a second camera is triggered to record images of a reachable food source when joint analysis of data from the EMG sensor and the motion sensor indicates that the person is consuming food.

In an example, a wearable food consumption monitoring device can comprise: eyeglasses worn by a person; wherein the eyeglasses further comprise at least two cameras; and wherein the eyeglasses further comprise an EMG sensor, a motion sensor, and an infrared sensor, wherein a first camera is triggered to record images along an imaging vector which points toward the person's mouth and a second camera is triggered to record images of a reachable food source when joint analysis of data from the EMG sensor, the motion sensor, and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise an motion sensor and a chewing sensor, wherein a first camera is triggered to record images along an imaging vector which points toward the person's mouth and a second camera is triggered to record images of a reachable food source when joint analysis of data from the motion sensor and the chewing sensor indicates that the person is consuming food.

In an example, a wearable food consumption monitoring device can comprise: eyeglasses worn by a person; wherein the eyeglasses further comprise at least two cameras; and wherein the eyeglasses further comprise an motion sensor, wherein a first camera is triggered to record images along an imaging vector which points toward the person's mouth and a second camera is triggered to record images of a reachable food source when analysis of data from the motion sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone) and a chewing sensor, wherein at least one camera is triggered to record images along an imaging vector which points toward the person's mouth when joint analysis of data from the sound sensor (e.g. microphone) and the chewing sensor indicates that the person is consuming food.

In an example, a wearable food consumption monitoring device can comprise: eyeglasses worn by a person; wherein the eyeglasses further comprise one or more cameras; and wherein the eyeglasses further comprise a sound sensor (e.g. microphone), a chewing sensor, and an infrared sensor, wherein at least one camera is triggered to record images along an imaging vector which points toward the person's mouth when joint analysis of data from the sound sensor (e.g. microphone), the chewing sensor, and the infrared sensor indicates that the person is consuming food. In another embodiment, a wearable food consumption monitoring device can comprise: eyeglasses worn by a person; wherein the eyeglasses further comprise one or more cameras; and wherein the eyeglasses further comprise a sound sensor (e.g. microphone), a chewing sensor, and an infrared sensor, wherein at least one camera is triggered to record images along an imaging vector which points toward the person's mouth when joint analysis of data from the sound sensor (e.g. microphone), the chewing sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone) and a motion sensor, wherein at least one camera is triggered to record images along an imaging vector which points toward the person's mouth when joint analysis of data from the sound sensor (e.g. microphone) and the motion sensor indicates that the person is consuming food.

In an example, a wearable food consumption monitoring device can comprise: eyeglasses worn by a person; wherein the eyeglasses further comprise one or more cameras; and wherein the eyeglasses further comprise a swallow sensor and an EEG sensor, wherein at least one camera is triggered to record images along an imaging vector which points toward the person's mouth when joint analysis of data from the swallow sensor and the EEG sensor indicates that the person is consuming food. In another embodiment, a wearable food consumption monitoring device can comprise: eyeglasses worn by a person; wherein the eyeglasses further comprise one or more cameras; and wherein the eyeglasses further comprise a swallow sensor, a chewing sensor, and an infrared sensor, wherein at least one camera is triggered to record images along an imaging vector which points toward the person's mouth when joint analysis of data from the swallow sensor, the chewing sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallow sensor, an EMG sensor, and an infrared sensor, wherein at least one camera is triggered to record images along an imaging vector which points toward the person's mouth when joint analysis of data from the swallow sensor, the EMG sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a swallowing sensor and an EMG sensor, wherein at least one camera is triggered to record images along an imaging vector which points toward the person's mouth when joint analysis of data from the swallowing sensor and the EMG sensor indicates that the person is consuming food. In another example, a wearable food consumption monitoring device can comprise: eyeglasses worn by a person; wherein the eyeglasses further comprise one or more cameras; and wherein the eyeglasses further comprise a swallowing sensor, an EMG sensor, and an infrared sensor, wherein at least one camera is triggered to record images along an imaging vector which points toward the person's mouth when joint analysis of data from the swallowing sensor, the EMG sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallowing sensor, a motion sensor, and an infrared sensor, wherein at least one camera is triggered to record images along an imaging vector which points toward the person's mouth when joint analysis of data from the swallowing sensor, the motion sensor, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise an accelerometer, wherein at least one camera is triggered to record images along an imaging vector which points toward the person's mouth when analysis of data from the accelerometer indicates that the person is consuming food.

In an example, a wearable food consumption monitoring device can comprise: eyeglasses worn by a person; wherein the eyeglasses further comprise one or more cameras; and wherein the eyeglasses further comprise an EEG sensor and an infrared sensor, wherein at least one camera is triggered to record images along an imaging vector which points toward the person's mouth when joint analysis of data from the EEG sensor and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an EEG sensor, an accelerometer, and an infrared sensor, wherein at least one camera is triggered to record images along an imaging vector which points toward the person's mouth when joint analysis of data from the EEG sensor, the accelerometer, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise an EMG sensor and a motion sensor, wherein at least one camera is triggered to record images along an imaging vector which points toward the person's mouth when joint analysis of data from the EMG sensor and the motion sensor indicates that the person is consuming food. In another example, a wearable food consumption monitoring device can comprise: eyeglasses worn by a person; wherein the eyeglasses further comprise one or more cameras; and wherein the eyeglasses further comprise an EMG sensor, a chewing sensor, and an infrared sensor, wherein at least one camera is triggered to record images along an imaging vector which points toward the person's mouth when joint analysis of data from the EMG sensor, the chewing sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise an EMG sensor, an EEG sensor, and an infrared sensor, wherein at least one camera is triggered to record images along an imaging vector which points toward the person's mouth when joint analysis of data from the EMG sensor, the EEG sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an motion sensor and an infrared sensor, wherein at least one camera is triggered to record images along an imaging vector which points toward the person's mouth when joint analysis of data from the motion sensor and the infrared sensor indicates that the person is consuming food.

I claim:

1. Smart eyewear for measuring food consumption comprising:
an eyewear frame worn by a person;
a camera on the eyewear frame which records food images when activated; and
a chewing sensor on the eyewear frame which detects when the person eats, wherein the camera is activated to record food images when data from the chewing sensor indicates that the person is eating.

2. A smart watch or wrist band for measuring food consumption comprising:
a smart watch or wrist band worn by a person;
a motion sensor on the smart watch or wrist band;
a camera on the smart watch or wrist band, wherein the camera is activated to record food images when data from the motion sensor indicates that the person is eating; and
a spectroscopic sensor on the smart watch or wrist band which analyzes the molecular and/or nutritional composition of food.

3. A wearable system for measuring food consumption comprising:
an eyewear frame worn by a person;
a chewing sensor on the eyewear frame which detects when the person eats;
a smart watch or wrist band worn by the person;
a motion sensor on the smart watch or wrist band;
a first camera on the eyewear frame which records food images when activated, wherein the first camera is activated to record food images when data from the chewing sensor and data from the motion sensor indicate that the person is eating; and
a second camera on the smart watch or wrist band which records food images when activated, wherein the second camera is activated to record food images when data from the chewing sensor and data from the motion sensor indicate that the person is eating.
